(19) United States
(12) Patent Application Publication
(10) Pub. No.: US 2026/0114832 A1
(43) Pub. Date: Apr. 30, 2026

(54) MACHINE LEARNING ENABLED ANALYSIS OF COMPUTED TOMOGRAPHY AND POSITRON EMISSION TOMOGRAPHY SCANS FOR CELL-OF-ORIGIN PREDICTION

(71) Applicant: Genentech, Inc., South San Francisco, CA (US)

(72) Inventor: Mohamed Skander JEMAA, San Francisco, CA (US)

(21) Appl. No.: 19/380,176

(22) Filed: Nov. 5, 2025

Related U.S. Application Data
(63) Continuation of application No. PCT/US2024/028160, filed on May 7, 2024.
(60) Provisional application No. 63/500,717, filed on May 8, 2023.

(51) Int. Cl. A61B 6/00; A61B 6/03; G06T 7/00; G06T 7/10; G06T 7/62; G06V 10/774; G06V 10/82; G06V 20/69

(52) U.S. Cl. CPC A61B 6/5217; A61B 6/032; A61B 6/037; A61B 6/5235; G06T 7/0016; G06T 7/10; G06T 7/62; G06V 10/774; G06V 10/82; G06V 20/695; G06V 20/698; G06T 2207/10081; G06T 2207/10104; G06T 2207/20076; G06T 2207/20081; G06T 2207/20084; G06T 2207/30024; G06T 2207/30096; G06V 2201/03

(57) ABSTRACT

A method may include receiving a positron emission tomography (PET) scan depicting a plurality of cancerous cells. One or more lesions depicted in the positron emission tomography (PET) scan may be identified. A cell-of-origin classification model may be applied to determine a cell-of-origin of each lesion depicted in the positron emission tomography (PET) scan. A molecular subtype profile for the plurality of cancerous cells depicted in the positron emission tomography (PET) may be determined based at least on the cell-of-origin of the individual lesions depicted in the positron emission tomography (PET) scan. The molecular subtype profile may include an overall cell-of-origin of the plurality of cancerous cells and/or a proportion of lesions having each possible cell-of-origin. Related systems and computer program products are also provided.

### CROSS REFERENCE TO RELATED APPLICATION

This application is a bypass continuation of International Patent Application No. PCT/US2024/028160, filed May 7, 2024, which claims the benefit of priority to U.S. Application No. 63/500,717, filed May 8, 2023, the disclosure of each of which is incorporated by reference herein in its entirety.

### TECHNICAL FIELD

The subject matter described herein relates generally to machine learning and more specifically to machine learning based technique for determining cell-of-origin (COO) based on positron emission tomography (PET) and computed tomography (CT) scans.

### INTRODUCTION

Medical imaging refers to techniques and processes for obtaining data characterizing a subject's internal anatomy and pathophysiology including, for example, images created by the detection of radiation either passing through the body (e.g. x-rays) or emitted by administered radiopharmaceuticals (e.g. gamma rays from intravenously administered radioactive tracers). By revealing internal anatomical structures obscured by other tissues such as skin, subcutaneous fat, and bones, medical imaging is integral to numerous medical diagnosis and/or treatments. Examples of medical imaging modalities include 2-dimensional imaging such as x-ray plain films, bone scintigraphy, and thermography. Examples of 3-dimensional imaging modalities include magnetic resonance imaging (MRI), computed tomography (CT), cardiac sestamibi scanning, and positron emission tomography (PET).

### SUMMARY

Systems, methods, and articles of manufacture, including computer program products, are provided for machine learning enabled analysis of positron emission tomography (PET) and computed tomography (CT) scans for determining the cell-of-origin (COO) of cancerous cells. Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

In one aspect, there is provided a method for machine learning enabled analysis of positron emission tomography (PET) and computed tomography (CT) scans for determining the cell-of-origin (COO) of cancerous cells. The method may include: comprising receiving a first positron emission tomography (PET) scan depicting a plurality of cancerous cells, identifying a first lesion depicted in the first PET scan, applying a cell-of-origin classification model to determine, based at least on the first lesion depicted in the first PET scan, a first cell-of-origin associated with the first lesion, and determining, based at least on the first cell-of-origin of the first lesion, a molecular subtype profile for the plurality of cancerous cells depicted in the first PET scan.

In another aspect, there is provided a system for machine learning enabled analysis of positron emission tomograph (PET) and computed tomography (CT) scans for determining the cell-of-origin (COO) of cancerous cells. The system may include at least one processor and at least one memory. The at least one memory may include program code that provides operations when executed by the at least one processor. The operations may include: comprising receiving a first positron emission tomography (PET) scan depicting a plurality of cancerous cells, identifying a first lesion depicted in the first PET scan, applying a cell-of-origin classification model to determine, based at least on the first lesion depicted in the first PET scan, a first cell-of-origin associated with the first lesion, and determining, based at least on the first cell-of-origin of the first lesion, a molecular subtype profile for the plurality of cancerous cells depicted in the first PET scan.

In another aspect, there is provided a computer program product for machine learning enabled analysis of positron emission tomography (PET) and computed tomography (CT) scans for determining the cell-of-origin (COO) of cancerous cells. The computer program product may include a non-transitory computer readable medium storing instructions that cause operations when executed by at least one data processor. The operations may include comprising receiving a first positron emission tomography (PET) scan depicting a plurality of cancerous cells, identifying a first lesion depicted in the first PET scan, applying a cell-of-origin classification model to determine, based at least on the first lesion depicted in the first PET scan, a first cell-of-origin associated with the first lesion, and determining, based at least on the first cell-of-origin of the first lesion, a molecular subtype profile for the plurality of cancerous cells depicted in the first PET scan.

In some variations of the methods, systems and non-transitory computer readable media, one or more of the following features can optionally be included in any feasible combination.

In some variations, the method may receive a first positron emission tomography (PET) scan depicting a plurality of cancerous cells, identify a first lesion depicted in the first PET scan, apply a cell-of-origin classification model to determine, based at least on the first lesion depicted in the first PET scan, a first cell-of-origin associated with the first lesion, and determine, based at least on the first cell-of-origin of the first lesion, a molecular subtype profile for the plurality of cancerous cells depicted in the first PET scan.

In some variations, the first cell-of-origin of the first lesion may include, for each possible cell-of-origin, a probability that one or more cancerous cells forming the first lesion is of that cell-of-origin.

In some variations, the method may identify a second lesion depicted in the first PET scan, apply the cell-of-origin classification model to determine, based at least on the second lesion depicted in the first PET scan, a second cell-of-origin associated with the second lesion, and determine, further based on the second cell-of-origin of the second lesion, the molecular subtype profile for the plurality of cancerous cells depicted in the first PET scan.

In some variations, the molecular subtype profile of the plurality of cancerous cells depicted in the first PET scan may include, for each possible cell-of-origin, a probability that an overall cell-of-origin of the plurality of cancerous cells is that cell-of-origin.

In some variations, the probability of the overall cell-of-origin of the plurality of cancerous cells being a particular cell-of-origin may be a maximum, a minimum, a mean, a median, and/or a mode of a respective probability of each of the first lesion and the second lesion having that particular cell-of-origin.

In some variations, the molecular subtype profile of the plurality of cancerous cells depicted in the first PET scan may include, for each possible cell-of-origin, a corresponding proportion of lesions having that cell-of-origin.

In some variations, the molecular subtype profile of the plurality of cancerous cells depicted in the first PET scan may be determined by at least generating a embedding sequence to include the first cell-of-origin of the first lesion and the second cell-of-origin of the second lesion, and applying a machine learning model to determine, based at least on the embedding sequence, an overall cell-of-origin of the plurality of cancerous cells in the first PET scan.

In some variations, the machine learning model may be a recurrent neural network.

In some variations, the method may extract, from the first PET scan, a volume including the first lesion identified in the first PET scan, and apply the cell-of-origin classification model to determine, based at least on the volume extracted from the first PET scan, the first cell-of-origin associated with the first lesion.

In some variations, the volume including the first lesion may be extracted by at least determining, within the first PET scan, a center of mass of the first lesion, and extracting, based at least on the center of mass of the first lesion, the volume.

In some variations, the volume may be a three-dimensional volume comprising a plurality of two-dimensional patches centered around the center of mass of the first lesion.

In some variations, the plurality of two-dimensional patches may include a plurality of axial patches or a plurality of coronal patches.

In some variations, the first lesion may be identified by at least applying a segmentation model to identify, within the first PET scan, a plurality of pixels corresponding to the first lesion.

In some variations, the method may extract, from the first PET scan, a plurality of features associated with the first lesion identified in the first PET scan, and apply the cell-of-origin classification model to determine, based at least on the plurality of features extracted from the first PET scan, the first cell-of-origin associated with the first lesion.

In some variations, the plurality of features may include a size of the first lesion, a shape of the first lesion, and/or a texture of the first lesion.

In some variations, the plurality of features may include one or more first-order statistics associated with one or more pixels depicting the first lesion in the first PET scan.

In some variations, the plurality of features may include a gray level co-occurrence matrix, a gray level size zone matrix, and/or a gray level run length matrix of one or more pixels depicting the first lesion in the first PET scan.

In some variations, the method may receive a computed tomography (CT) scan from a same timepoint as the first PET scan, identify the first lesion depicted in the CT scan, and apply the cell-of-origin classification model to determine, based on the first lesion depicted in the first PET scan and the CT scan, the first cell-of-origin associated with the first lesion.

In some variations, each pixel included in the CT scan may be associated with an intensity value corresponding to a tissue density or an x-ray attenuation.

In some variations, each pixel in the first PET scan may be associated with an intensity value corresponding to a level of metabolic activity.

In some variations, the method may determine, based on at least one of the CT scan and the first PET scan, a tumor mask corresponding to the first lesion, and apply the cell-of-origin classification model to determine, further based at least on the tumor mask, the cell-of-origin of the first lesion.

In some variations, the method may determine, based on at least one of the CT scan and the first PET scan, an organ mask corresponding to one or more organs depicted in the CT scan and the first PET scan, and apply the cell-of-origin classification model to determine, further based at least on the organ mask, the cell-of-origin of the first lesion.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to fluorodeoxyglucose avid (FDG-avid) cancers such as non-Hodgkin lymphoma (NHL), it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a machine learning based medical imaging analysis system, in accordance with some example embodiments:

FIG. 2A depicts a schematic diagram illustrating an example of a process for machine learning enabled cell-of-origin (COO) prediction, in accordance with some example embodiments:

FIG. 2B depicts a schematic diagram illustrating another example of a process for machine learning enabled cell-of-origin (COO) prediction, in accordance with some example embodiments:

FIG. 3 depicts a flowchart illustrating an example of a process for machine learning enabled cell-of-origin prediction, in accordance with some example embodiments:

FIG. 4A depicts a flowchart illustrating another example of a process for machine learning enabled cell-of-origin prediction, in accordance with some example embodiments:

FIG. 4B depicts a flowchart illustrating another example of a process for machine learning enabled cell-of-origin prediction, in accordance with some example embodiments:

FIG. 5A depicts graphs illustrating a comparison of various performance metrics for different implementations of a cell-of-origin classification model across different clinical datasets, in accordance with some example embodiments:

FIG. 5B depicts graphs illustrating a comparison of various performance metrics for different implementations of a cell-of-origin classification model across different clinical datasets, in accordance with some example embodiments:

FIG. 5C depicts graphs illustrating a comparison of various performance metrics for different implementations of a cell-of-origin classification model across different clinical datasets, in accordance with some example embodiments:

FIG. 5D depicts graphs illustrating a comparison of various performance metrics for different implementations of a cell-of-origin classification model across different clinical datasets, in accordance with some example embodiments:

FIG. 6A depicts graphs illustrating a comparison of the receiver operating characteristic (ROC) curves of different molecular subtype classifications made by a cell-of-origin classification model across different test sets, in accordance with some example embodiments:

FIG. 6B depicts graphs illustrating a comparison of the receiver operating characteristic (ROC) curves of different molecular subtype classifications made by a cell-of-origin classification model across different test sets, in accordance with some example embodiments:

FIG. 7A depicts graphs illustrating the importance of various radiomic features for different implementations of a cell-of-origin classification model, in accordance with some example embodiments:

FIG. 7B depicts graphs illustrating the importance of various radiomic features for different implementations of a cell-of-origin classification model, in accordance with some example embodiments:

FIG. 7C depicts graphs illustrating the importance of various radiomic features for different implementations of a cell-of-origin classification model, in accordance with some example embodiments; and

FIG. 8 depicts a block diagram illustrating an example of a computing system, in accordance with some example embodiments.

When practical, similar reference numbers denote similar structures, features, or elements.

### DETAILED DESCRIPTION

Heterogenous diseases, including cancers such as diffuse large B-cell lymphoma (DLBCL), are associated with complex etiological factors as well as diverse molecular and cellular dysfunctions. Biological insights into disease heterogeneity may play a critical role in patient prognosis and treatment selection. For example, in some cases, molecular subtypes derived based on the cell-of-origin of cancerous cells may serve as a crucial biomarker for predicting patient response to therapy and survival. In the case of diffuse large B-cell lymphoma (DLBCL), patients with the germinal center B-cell (GCB) molecular subtype tend to have a better prognosis than patients with the non-germinal center B-cell (non-GCB) or activated B-cell (ABC) molecular subtype. Accordingly, identifying a patient's molecular subtype may be imperative for identifying effective therapeutic options.

Despite genetic and phenotypic variations, heterogeneous diseases, such as diffuse large B-cell lymphoma, will often have similar clinical presentations. Accordingly, determining the molecular subtype of a heterogeneous disease, such as the cell-of-origin of a cancer (e.g., diffuse large B-cell lymphoma and/or the like), generally requires expensive procedures that are not commonplace in standard clinical practice. For example, conventional techniques for determining cell-of-origin (COO), such as ribonucleic acid sequencing (RNASeq) and the immunohistochemistry (IHC) based Hans algorithm, rely on invasive assays to extract tumor tissue. Subsequent analytical techniques suffer from either limited availability (e.g., genetic expression profiling (GEP)) or limited performance, limited reproducibility, and high inter-reader variability (IHC). Consequently, with conventional techniques for determining cell-of-origin, accurate and precise molecular subtype determination remains an onerous task that is exceeds the resources of typical cancer patients. To overcome the aforementioned limitations of conventional molecular subtyping techniques, an analysis controller may apply a machine learning based cell-of-origin classification model to determine, based at least on one or more medical images depicting a plurality of cancerous cells, the cell-of-origin of the plurality of cancerous cells. For instance, in some cases, the machine learning based cell-of-origin classification model may be applied to a positron emission tomography (PET) scan and/or a computed tomography (CT) scan depicting the plurality of cancerous cells. Medical images, such as positron emission tomography (PET) scans and computed tomography (CT) scans, may be obtained non-invasively and are acquired routinely for cancer patients. Accordingly, unlike conventional techniques, the cell-of-origin classification model described herein may be capable of making an accurate and precise cell-of-origin based molecular subtype determination non-invasively and with minimal added resources.

Various modalities of medical imaging may be applied to obtain data characterizing a subject's internal anatomy as well as pathophysiology. Computed tomography (CT) is an example of a three-dimensional imaging modality in which a series of X-rays are captured to create cross-sectional images (e.g., patches, slices, and/or the like) of the bones, blood vessels, and soft tissues inside the body. A computed tomography scan may be a three-dimensional volume formed by a series of two-dimensional images in which each pixel is associated with an intensity value indicative of a tissue density or x-ray attenuation at the corresponding location in the subject's body. Another example of a three-dimensional imaging modality is positron emission tomography (PET), which captures radioactivity signals indicative of cellular metabolic activities inside the subject's body. A positron emission tomography scan may be a three-dimensional volume formed by a series of two-dimension images in which each pixel is associated with an intensity value indicative of the level of cellular metabolic activity (e.g., glucose uptake) at the corresponding location in the subject's body. In some cases, a single gantry incorporating a positron emission tomography (PET) scanner and a computed tomography (CT) scanner may be capable of acquiring positron emission tomography (PET) scans and computed tomography (CT) scans during a same session. The resulting positron emission tomography (PET) scan and computed tomography (CT) scan may be combined into a single superposed (e.g., co-registered) image (e.g., a PET-CT scan) in which the spatial distribution of metabolic activities depicted in the positron emission tomography (PET) scan is aligned with the anatomical structures depicted in the computed tomography (CT) scan.

In some example embodiments, the analysis controller may determine, based at least on a positron emission tomography (PET) scan, the cell-of-origin of a plurality of cancerous cells depicted in the positron emission tomography (PET) scan. The plurality of cancerous cells may correspond to one or more lesions depicted in the positron emission tomography (PET) scan. Accordingly, in some cases, the analysis controller may apply a cell-of-origin classification model to determine, based at least on a first lesion depicted in the positron emission tomography (PET) scan, a first cell-of-origin of the first lesion. Moreover, in some cases, the cell-of-origin classification model may be applied to determine, based at least on a second lesion depicted in the positron emission tomography (PET) scan, a second cell-of-origin of the second lesion. A molecular subtype profile for the plurality of cancerous cells depicted in the positron emission tomography (PET) scan may be determined based on the first cell-of-origin of the first lesion and the second cell-of-origin of the second lesion. For example, in some cases, the molecular subtype profile of the plurality of cancerous cells may include an overall cell-of-origin of the plurality of cancerous cells determined based on the first cell-of-origin of the first lesion and the second cell-of-origin of the second lesion. Alternatively and/or additionally, the molecular subtype profile of the plurality of cancerous cells may include a proportion of different cells-of-origin present in the plurality of cancerous cells. This proportion of different cells-of-origin may be determined based at least on the first cell-of-origin of the first lesion and the second cell-of-origin of the second lesion.

In some example embodiments, the cell-of-origin classification model may determine, for each of the first lesion and the second lesion, a respective probability of the constituent cancerous cells having each of a plurality of different possible cells-of-origin. In the case of diffuse large B-cell lymphoma (DLBCL), for example, the cell-of-origin classification model may determine, for each of the first lesion and the second lesion, a first probability of the constituent cancerous cells having a first cell-of-origin (e.g., germinal center B cell (GCB)) and a second probability of the constituent cancerous cells having a second cell-of-origin. The overall cell-of-origin of the plurality of cancerous cells may include a maximum, a minimum, a mean, a median, and/or a mode of the first probability of each of the lesions having the first cell-of-origin. In some cases, the overall cell-of-origin of the plurality of cancerous cells may also include a maximum, a minimum, a mean, a median, and/or a mode of the second probability of each of the lesions having the second cell-of-origin. Furthermore, in some cases, the overall cell-of-origin of the plurality of cancerous cells may be determined based on one or more other characteristics of the first lesion and the second lesion including, for example, dimensions (e.g., length, width, volume), location (e.g., spatial coordinates, distance to other lesions), and/or the like. In some cases, the probability of each lesion having a particular cell-of-origin may be weighted, for example, based on the one or more additional characteristics, when determining the probability of the overall cell-of-origin being that particular cell-of-origin. Moreover, in some cases, the overall cell-of-origin of the plurality of cancerous cells may be identified as being a particular cell-of-origin (e.g., germinal center B cell (GCB) or activated B cell (ABC) (or non-germinal center B-cell (non-GCB)) if the probability of a threshold quantity (e.g., percentage, ratio, and/or the like) of lesions being associated with that particular cell-of-origin satisfies one or more thresholds.

In some example embodiments, the overall cell-of-origin of the plurality of cancerous cells depicted in the positron emission tomography (PET) scan may be determined by at least applying a machine learning model (e.g., a neural network such as a recurrent neural network (RNN) and/or the like) that operates a embedding sequence that includes, for each lesion present in the positron emission tomography (PET) scan, the probability of the constituent cancerous cells having each possible cell-of-origin in order to determine the overall cell-of-origin of the plurality of cancerous cells. In some cases, the embedding sequence may also include one or more other characteristics of each lesion such as, for example, one or more dimensions of each lesion (e.g., length, width, volume), the location of each lesion (e.g., spatial coordinates, distance to other lesions), and/or the like.

Alternatively and/or additionally, the molecular subtype profile of the plurality of cancerous cells depicted in the positron emission tomography (PET) scan may include one or more metrics indicative of a heterogeneity and/or a uniformity between the different cells-of-origin included in the plurality of cancerous cells. of the first lesion and the second cell-of-origin of the second lesion. For example, in some cases, the molecular subtype profile of the plurality of cancerous cells may include a proportion (e.g., percentage, ratio, and/or the like) of lesions identified as having each possible cell-of-origin. In the case of diffuse large B-cell lymphoma (DLBCL), for instance, the molecular subtype profile of the plurality of cancerous cells may include a first proportion of lesions identified as germinal center B cell (GCB) and a second proportion of lesions identified as activated B cell (ABC) (or non-germinal center B-cell (non-GCB)).

In some example embodiments, the cell-of-origin of each lesion depicted in the positron emission tomography (PET) scan may be determined based on a corresponding volume extracted from the positron emission tomography (PET) scan. For example, in some cases, a volume extracted from the positron emission tomography (PET) scan may be a three-dimensional volume formed from a series of two-dimensional patches (e.g., axial patches, coronal patches, and/or the like). Accordingly, the analysis controller may extract, from the positron emission tomography (PET) scan, a first volume centered around a first center of mass of the first lesion and a second volume centered around a second center of mass of the second lesion. In some cases, the analysis controller may apply the cell-of-origin classification model to determine, based at least on the first volume extracted from the positron emission tomography (PET) scan, the first cell-of-origin of the first lesion. Moreover, in some cases, the analysis controller may apply the cell-of-origin classification model to determine, based at last on the second volume extracted from the positron emission tomography (PET) scan, the second cell-of-origin of the second lesion.

In some example embodiments, the cell-of-origin of each lesion depicted in the positron emission tomography (PET) scan may be determined based on one or more corresponding radiomic features extracted from the positron emission tomography (PET) scan. For example, in some cases, the analysis controller may extract, for each lesion depicted in the positron emission tomography (PET) scan, the size of the lesion, the shape of the lesion, and/or the texture of the lesion. Alternatively and/or additionally, the analysis controller may extract, for each lesion depicted in the positron emission tomography (PET) scan, one or more first-order statistics, a gray level co-occurrence matrix, a gray level size zone matrix, and/or a gray level run length matrix of one or more pixels of the positron emission tomography (PET) scan depicting the lesion. In some cases, the analysis controller may apply the cell-of-origin classification model to determine, based at least on a first plurality of features of the first lesion extracted from the positron emission tomography (PET) scan, the first cell-of-origin of the first lesion. Moreover, in some cases, the analysis controller may apply the cell-of-origin classification model to determine, based at least on a second plurality of features of the second lesion extracted from the second positron emission tomography (PET) scan, the second cell-of-origin of the second lesion.

In some example embodiments, the analysis controller may determine the molecular subtype (or the overall cell-of-origin) of the plurality of cancerous cells based on multiple modalities of medical images from the same timepoint. For example, in some cases, the molecular subtype (or the overall cell-of-origin) of the plurality of cancerous cells may be determined based on the positron emission tomography (PET) scan depicting the plurality of cancerous cells as well as a computed tomography (CT) from a same timepoint. That is, in some cases, the first cell-of-origin of the first lesion and the second cell-of-origin of the second lesion may be determined based on the positron emission tomography (PET) scan as well as the corresponding computed tomography (CT) scan. Moreover, in some cases, the first cell-of-origin of the first lesion and the second cell-of-origin of the second lesion may be determined by at least applying the cell-of-origin classification model to a tumor mask of the first lesion and the second lesion determined based on the positron emission tomography (PET) scan and the corresponding computed tomography (CT) scan. Alternatively and/or additionally, the first cell-of-origin of the first lesion and the second cell-of-origin of the second lesion may be determined by at least applying the cell-of-origin classification model to an organ mask of one or more organs determined based on the positron emission tomography (PET) scan and the corresponding computed tomography (CT) scan.

In some example embodiments, the analysis controller may determine a molecular subtype profile for the plurality of cancerous cells based on positron emission tomography (PET) scans from multiple timepoints. For example, in some cases, the analysis controller may apply the cell-of-origin classification model to determine, based at least on the first lesion depicted in a first positron emission tomography (PET) scan from a first timepoint and a second positron emission tomography (PET) scan from a second timepoint, the first cell-of-origin of the first lesion. Furthermore, in some cases, the analysis controller may apply the cell-of-origin classification model to determine the first cell-of-origin of the first lesion based on the first lesion depicted in a

first computed tomography (CT) scan from a same timepoint as the first positron emission tomography (PET) scan and a second computed tomography (CT) scan from a same timepoint as the second positron tomography (PET) scan. In some cases, the molecular subtype profile of the plurality of cancerous cells may include an overall cell-of-origin determined based at least on the first cell-of-origin of the first lesion and the second cell-of-origin of the second lesion. Alternatively and/or additionally, the molecular subtype profile of the plurality of cancerous cells may include a proportion of the different cells-of-origin present in the positron emission tomography (PET) scan determined based at least on the first cell-of-origin of the first lesion and the second cell-of-origin of the second lesion.

In some example embodiments, the analysis controller may further determine, based on the molecular subtype profile associated with the plurality of cancerous cells depicted in the positron emission tomography (PET) scan, a disease diagnosis, a disease prognosis, a disease progress, a treatment, and/or a treatment response for a patient associated with the positron emission tomography (PET) scan. For example, in some cases, the plurality of cancerous cells may be associated with diffuse large B-cell lymphoma (DLBCL). Accordingly, the cell-of-origin classification model may be trained to determine, for each lesion present in the positron emission tomography (PET) scan, a first probability of the constituent cancerous cells being germinal center B cell (GCB) and a second probability of the constituent cancerous cells being activated B cell (ABC) (or non-germinal center B cell (non-GCB)). The molecular subtype profile of the plurality of cancerous cells may be determined based on the first probability of each lesion being germinal center B cell (GCB) and the second probability of the constituent cancerous cells being activated B cell (ABC) (or non-germinal center B cell (non-GCB)). For instance, in some cases, the molecular subtype profile of the plurality of cancerous cells may include an overall cell-of-origin and/or a proportion of the different cells-of-origin present in plurality of cancerous cells depicted in the positron emission tomography (PET) scan.

In some example embodiments, the cell-of-origin classification model may include one or more of an artificial neural network (ANN) (e.g., a vision transformer model such as a vision transformer model with shifted patch tokenization and locality self-attention), a tree-based classifier (e.g., a gradient boosted decision tree, a random forest, an extreme gradient boosted decision tree (XGBoost)), a ridge classifier, and/or the like. The cell-of-origin classification model may be trained based on a training set that includes one or more annotated training samples. For example, in some cases, the training set may be generated to include a first training sample having a first volume including a lesion depicted in a positron emission tomography (PET) scan and a first ground-truth annotation of a cell-of-origin of the lesion. Furthermore, in some cases, the training set may be generated to include a second training sample having a second volume including the same lesion depicted in the positron emission tomography (PET) scan and a second ground-truth annotation of the cell-of-origin of the lesion. The second volume may be generated by modifying the first volume, for example, by one or more of normalizing, rotating, flipping, and changing a zoom of the first volume. In some cases, the modifying of the first volume may be limited to one or more slices of the first volume that are within a threshold distance of a center of mass of the lesion included in the first volume.

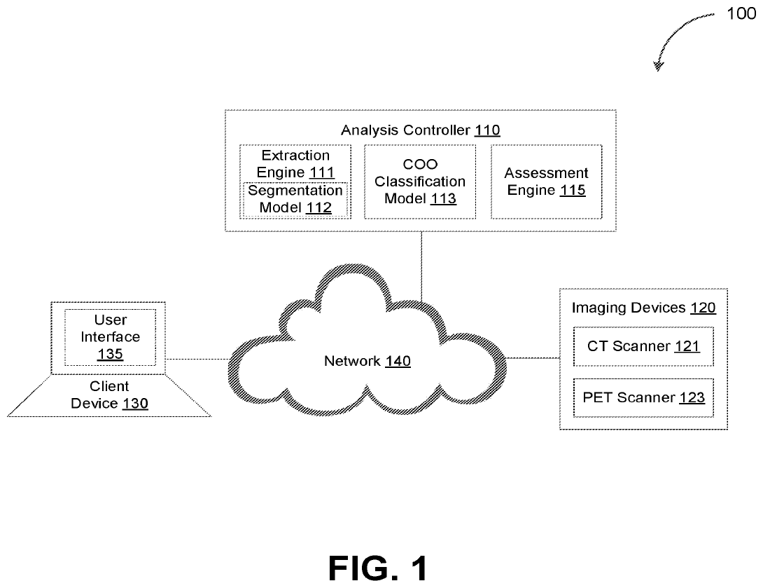

FIG. 1 depicts a system diagram illustrating an example of a machine learning based medical imaging analysis system 100, in accordance with some example embodiments. Referring to FIG. 1, the machine learning based medical imaging analysis system 100 may include an analysis controller 110, one or more imaging devices 120, and a client device 130. As shown in FIG. 1, the analysis controller 110, the one or more imaging devices 120, and the client device 130 may be communicatively coupled via a network 140. The one or more imaging devices 120 may include, for example, a computed tomography (CT) scanner 121 and a positron emission tomography (PET) scanner 123. The client device 130 may be a processor-based device including, for example, a smartphone, a tablet computer, a wearable apparatus, a virtual assistant, an Internet-of-Things (IoT) appliance, and/or the like. The network 140 may be a wired network and/or a wireless network including, for example, a wide area network (WAN), a local area network (LAN), a virtual local area network (VLAN), a public land mobile network (PLMN), the Internet, and/or the like.

In the example shown in FIG. 1, the analysis controller 110 may include an extraction engine 111 including a segmentation model 112, a cell-of-origin classification model 113, and an analysis engine 115. In some example embodiments, the analysis controller 110 may determine, based at least on a positron emission tomography (PET) scan depicting a plurality of cancerous cells, a molecular subtype profile of the one or more lesions formed by the plurality of cancerous cells. In some cases, the analysis controller 110 may further determine the molecular subtype profile of the one or more lesions based on a computed tomography (CT) scan from a same timepoint. The positron emission tomography (PET) scan and the computed tomography (CT) scan may be generated by the one or more imaging devices 120 (e.g., the computed tomography scanner 121, the positron emission tomography scanner (PET) scanner 123, and/or the like).

In some cases, the analysis controller 110 may apply the cell-of-origin classification model 113, which may be trained to determine the cell-of-origin of each lesion. For example, the cell-of-origin classification model 113 may be applied to determine, based on the positron emission tomography (PET) scan and, in some cases, the computed tomography

(CT) scan from the same timepoint, a first cell-of-origin of a first lesion. Furthermore, the cell-of-origin classification model 113 may be applied to determine, based on the positron emission tomography (PET) scan and, in some cases, the computed tomography (CT) scan from the same timepoint, a second cell-of-origin of a second lesion. As will be described in more detail, in some cases, the first cell-of-origin of the first lesion and the second cell-of-origin of the second lesion may be determined based on volumes including the first lesion and the second lesion extracted by the extraction engine 111. Alternatively, the first cell-of-origin of the first lesion and the second cell-of-origin of the second lesion may be determined based on features extracted by the extraction engine 111. Moreover, the assessment engine 115 may determine the molecular subtype profile based on the first cell-of-origin of the first lesion and the second cell-of-origin of the second lesion.

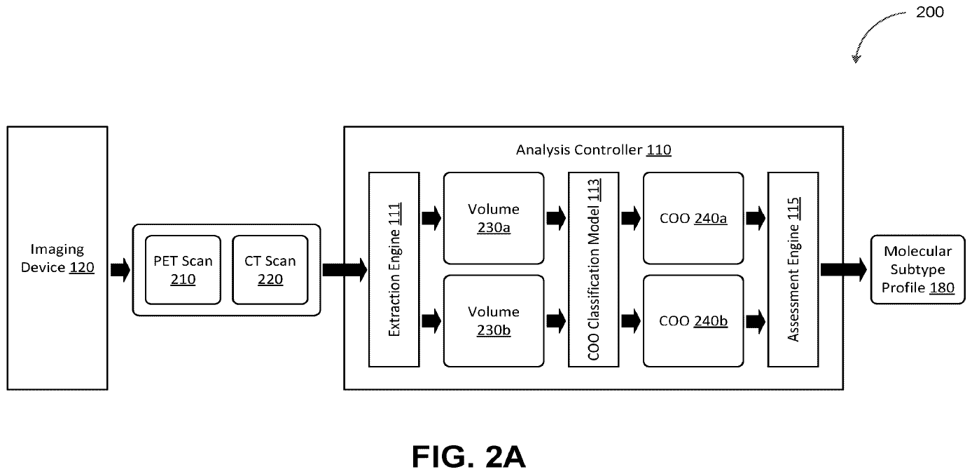

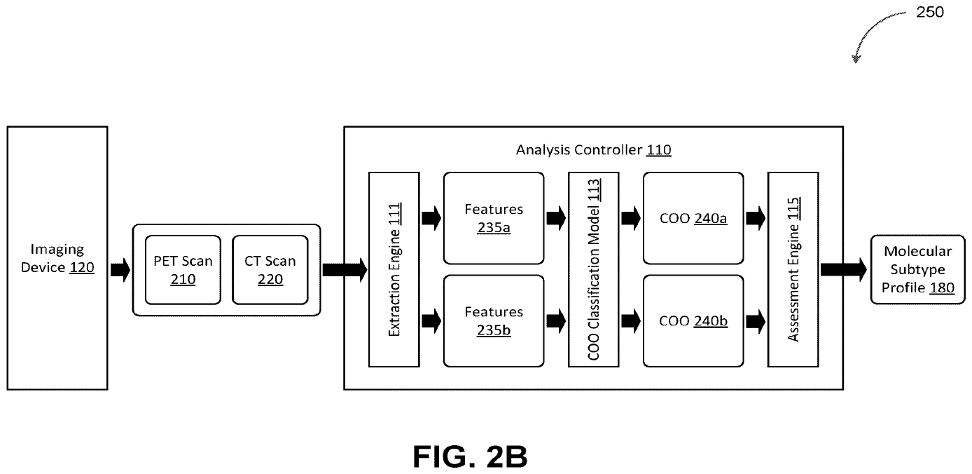

FIG. 2A depicts a schematic diagram illustrating an example of a process 200 for machine learning enabled cell-of-origin prediction, in accordance with some example embodiments. Referring to FIG. 2A, the analysis controller 110 may receive, from the one or more imaging devices 120, a positron emission tomography (PET) scan 210 and, in some cases, a computed tomography (CT) scan 220. For example, in some cases, the analysis controller 110 may determine, based on the positron emission tomography (PET) scan 210, a molecular subtype profile 180 of a plurality of cancerous cells depicted in the positron emission tomography (PET) scan 210. Alternatively, the analysis controller 110 may determine the molecular subtype profile 180 based on the positron emission tomography (PET) scan 210 as well as the computed tomography (CT) scan 220. In some cases, the positron emission tomography (PET) scan 210 and the computed tomography (CT) scan 220 may be from a same timepoint. Accordingly, the positron emission tomography (PET) scan 210 may be superimposed (or co-registered) with the computed tomography (CT) scan 220 such that each pixel in the positron emission tomography (PET) scan 210 is mapped to a corresponding pixel in the computed tomography (CT) scan 220.

In some example embodiments, the analysis controller 110 may determine the molecular subtype profile 180 of the plurality of cancerous cells depicted in the positron emission tomography (PET) scan 210 and the computed tomography (CT) scan 220 based on a cell-of-origin of each individual lesion included in one or more volumes extracted from the positron emission tomography (PET) scan 210 and the computed tomography (CT) scan 220. For example, the plurality of cancerous cells depicted in the positron emission tomography (PET) scan 210 and the computed tomography (CT) scan 220 may form one or more lesions.

Accordingly, each volume extracted from the positron emission tomography (PET) scan 210 and, in some cases, the co-registered computed tomography (CT) scan 220 may be a three-dimensional volume having a plurality of two-dimensional patches (e.g., 96 pixels by 96 pixels or a different size) centered around a center of mass of a corresponding lesion. In the example of the process 200 shown in FIG. 2A, the analysis controller 110 may include an extraction engine 111 configured to extract, from the positron emission tomography (PET) scan and, in some cases, the co-registered computed tomography (CT) scan 220, a first volume 230a including a first lesion and a second volume 230b including a second lesion.

In some example embodiments, the extraction engine 111 may extract the first volume 230a and the second volume 230b by at least identifying, within the positron emission tomography (PET) scan 210 and, in some cases, the co-registered computed tomography (CT) scan 220, a corresponding lesion. For example, in some cases, the extraction engine 111 may segment (e.g., by applying the segmentation model 112 such as an artificial neural network and/or the like) to identify, within the positron emission tomography (PET) scan 210 and the computed tomography (CT) scan 220, a first plurality of pixels corresponding to the first lesion and a second plurality of pixels corresponding to the second lesion. Moreover, the extraction engine 111 may determine a first center of mass of the first lesion and a second center of mass of the second lesion. The first volume 230a extracted from the positron emission tomography (PET) scan 210 and, in some cases, the co-registered computed tomography (CT) scan 220, may include a first plurality of two-dimensional patches (e.g., axial patches, coronal patches, and/or the like) centered around the first center of mass of the first lesion. Meanwhile, the second volume 230b may include a second plurality of two-dimensional patches (e.g., axial patches, coronal patches, and/or the like) centered around the second center of mass of the second lesion.

Referring again to FIG. 2A, in some example embodiments, the analysis controller 110 may apply a cell-of-origin classification model 113 to determine, based at least on the first volume 230a, a first cell-of-origin 240a of the first lesion included in the first volume 230a. Furthermore, in the example of the process 200 shown in FIG. 2A, the analysis controller 110 may apply the cell-of-origin classification model 113 to determine, based at least on the second volume 230b, a second cell-of-origin 240b of the second lesion included in the second volume 230b. In some cases, the first cell-of-origin 240a of the first lesion may include, for each possible cell-of-origin (e.g., cell-of-origin label), a probability of the first lesion depicted in the first volume 230a having the corresponding cell-of-origin while the second cell-of-origin 240b of the second lesion may include, for each possible cell-of-origin (e.g., cell-of-origin label), a probability of the second lesion depicted in the second volume 230b having the corresponding cell-of-origin. For example, in the case of diffuse large B-cell lymphoma (DLBCL), the first cell-of-origin 240a may include a first probability of the first lesion being germinal center B cell (GCB) and/or a second probability of the first lesion being activated B cell (ABC) (or non-germinal center B cell (non-GCB). Similarly, the second cell-of-origin 240b may include a third probability of the second lesion being germinal center B cell (GCB) and/or a fourth probability of the second lesion being activated B cell (ABC) (or non-germinal center B cell (non-GCB). As shown in FIG. 2A, the analysis controller 110 may further include an assessment engine 115 that determines, based at least on the first cell-of-origin 240a of the first lesion and the second cell-of-origin 240b of the second lesion, the molecular subtype profile 180 of the plurality of cancerous cells depicted in the positron emission tomography (PET) scan 210 and the computed tomography (CT) scan 220. For instance, in the case of diffuse large B-cell lymphoma (DLBCL), the molecular subtype profile 180 may be determined based at least on the first probability of the first lesion being germinal center B cell (GCB), the second probability of the first lesion being activated B cell (ABC) (or non-germinal center B cell (non-GCB), the third probability of the second lesion being germinal center B cell (GCB), and/or the fourth probability of the second lesion being activated B cell (ABC) (or non-germinal center B cell (non-GCB).

In some example embodiments, instead of extracting one or more volumes centered around a center of mass of one or more corresponding lesions, the extraction engine 111 may extract, from the positron emission tomography (PET) scan 210 and, in some cases, the co-registered computed tomography (CT) scan 220, one or more features of each lesion. To further illustrate, FIG. 2B depicts another example of the process 250 in which the extraction engine 111 extracts, from the positron emission tomography (PET) scan 210 and, in some cases, the co-registered computed tomography (CT) scan 220, a first plurality of features 235a of the first lesion and a second plurality of features 235b of the second lesion. The first plurality of features 235a and the second plurality of features 235b may include one or more radiomic features. For example, in some cases, the first plurality of features 235a and the second plurality of features 235b may each include a size, a shape, and/or a texture of the corresponding lesion.

Alternatively and/or additionally, the first plurality of features 235a and the second plurality of features 235b may also include, for each corresponding lesion, one or more first-order statistics such as a range, a maximum, a minimum, a median, a mode, and/or a mean pixel value of the one or more pixels depicting the lesion in the positron emission tomography (PET) scan 210 and, in some cases, the co-registered computed tomography (CT) scan 220. For the positron emission tomography (PET) scan 210, these first-order statistics may correspond to the first-order statistics of the level of metabolic activity (e.g., a standard uptake value (SUV)) exhibited by the lesion. Meanwhile, for the computed tomography (CT) scan 220, these first-order statistics may correspond to the first-order statistics of the tissue density (or x-ray attenuation) observed across the lesion. In some cases, the first plurality of features 235a and the second plurality of features 235b may further include, for the corresponding lesion, a gray level co-occurrence matrix, a gray level size zone matrix, and/or a gray level run length matrix of the one or more pixels depicting the lesion in the positron emission tomography (PET) scan 210 and, in some cases, the co-registered computed tomography (CT) scan 220.

Referring again to FIG. 2B, the analysis controller 110 may apply the cell-of-origin classification model 113 to determine, based at least on the first plurality of features 23Sa, the first cell-of-origin 240a of the first lesion. Furthermore, the analysis controller 110 may apply the cell-of-origin classification model 113 to determine, based at least on the second plurality of features 235b, the second cell-of-origin 240b of the second lesion. As shown in FIG. 2B, the assessment engine 115 may then determine, based at least on the first cell-of-origin 240a of the first lesion and the second cell-of-origin 240b of the second lesion, the molecular subtype profile 180 of the plurality of cancerous cells depicted in the positron emission tomography (PET) scan 210 and the computed tomography (CT) scan 220.

In some example embodiments, the assessment engine 115 may determine the molecular subtype profile 180 of the plurality of cancerous cells to include an overall cell-of-origin determined based on the cell-of-origin of each lesion present in the positron emission tomography (PET) scan 210 and the computed tomography (CT) scan 220. For example, in FIGS. 2A-B, the overall cell-of-origin included in the molecular subtype profile 180 may be determined based on the first cell-of-origin 240a of the first lesion and the second cell-of-origin 240b of the second lesion. In some cases, the overall cell-of-origin of the plurality of cancerous cells may be determined based on Equation (1) below. For instance, the overall cell-of-origin of the plurality of cancerous cells may include, for each possible cell-of-origin (e.g., cell-of-origin label) m, a corresponding probability Pₘ that the overall cell-of-origin is of the cell-of-origin m. That is, in some cases, the overall cell-of-origin of the plurality of cancerous cells may be denoted as (P₀, . . . , Pₘ). In the case of diffuse large B-cell lymphoma (DLBCL), the overall cell-of-origin of the plurality of cancerous cells may be denoted as (P_{GCB}, P_{ABC}), wherein P_{GCB} denotes the probability that the overall cell-of-origin is germinal B-cell and P_{ABC} denotes the probability that the overall cell-of-origin is activated B-cell (or non-germinal center B-cell (non-GCB)).

According to Equation (1) below, the probability Pm associated with the cell-of-origin m may be determined based on the probability Pₙ of each lesion n being of the cell-of-origin m. For example, Equation (1) shows one example where the probability Pm associated with the cell-of-origin m is the mean (or average) of the individual probabilities (p₀, . . . , pₙ). Alternatively, the probability Pₘ associated with the cell-of-origin m may be the maximum, minimum, median, and/or mode of the probabilities (p₀, . . . pₙ) associated with the individual lesions n. Equation (1) below further shows that, in some cases, the probability pₙ of each lesion n may be weighted by a weight wₙ corresponding to one or more additional characteristics of the lesion n. Examples of such characteristics include one or more dimensions of each lesion (e.g., length, width, volume), the location of each lesion (e.g., spatial coordinates, distance to other lesions), and/or the like. In some cases, the overall cell-of-origin of the plurality of cancerous cells may be identified as being a particular cell-of-origin if the probability of a threshold quantity (e.g., percentage, ratio, and/or the like) of lesions being associated with that particular cell-of-origin satisfies one or more thresholds. For instance, in the case of diffuse large B-cell lymphoma (DLBCL), the overall cell-of-origin of the plurality of cancerous cells may be identified as germinal center B cell (GCB) if more than 25% of lesions depicted in the positron emission tomography (PET) scan 210 and the computed tomography (CT) scan 220 exhibits a greater than 50% likelihood of being germinal center B-cell (GCB).

$${P}_{m} = \frac{{Σ}_{i = 0}^{i = n} {w}_{i} {p}_{i}}{n} \qquad ( 1 )$$

In some cases, the assessment engine 115 may apply a machine learning model (e.g., a recurrent neural network and/or the like) to determine the overall cell-of-origin of the plurality of cancerous cells. For example, the assessment engine 115 may generate an embedding sequence to include, for each lesion, the probability of the constituent cancerous cells having each possible cell-of-origin. In the case of diffuse large B-cell lymphoma (DLBCL), the embedding sequence may include, for each lesion, a first probability of the constituent cancerous cells being germinal B-cells (GCB) and a second probability of the constituent cancerous cells being activated B-cells (ABC) (or non-germinal B-cells (non-GCB)). The machine learning model (e.g., a recurrent neural network and/or the like) may be applied to the embedding sequence to determine the overall cell-of-origin for inclusion in the molecular subtype profile 180 of the plurality of cancerous cells. In some cases, the embedding sequence may be generated to include one or more additional characteristics of each lesion including, for example, the dimensions of each lesion (e.g., length, width, volume), the location of each lesion (e.g., spatial coordinates, distance to other lesions), and/or the like.

In some cases, in addition to or instead of the overall cell-of-origin, the assessment engine 115 may determine the molecular subtype profile 180 to include one or more metrics indicative of a heterogeneity and/or a uniformity of the cell-of-origin of the different lesions (e.g., the first cell-of-origin 240a of the first lesion and the second cell-of-origin 240b of the second lesion). For example, in some cases, the molecular subtype profile 180 may include, for each possible cell-of-origin, a corresponding proportion (e.g., percentage, ratio, and/or the like) of lesions identified as exhibiting the cell-of-origin. In the case of diffuse large B-cell lymphoma (DLBCL), the molecular subtype profile 180 may include a first proportion of lesions identified as being germinal center B-cell (GCB) and a second proportion of lesions identified as being activated B-cell (ABC) (or non-germinal center B-cell (non-GCB)).

In some example embodiments, the cell-of-origin classification model 113 may include one or more machine learning models trained to determine the cell-of-origin associated with a lesion based on a volume including the lesion and/or one or more features of the lesion extracted from a positron emission tomography (PET) scan and, in some cases, a computed tomography (CT) scan from a same timepoint. For example, in some cases, the cell-of-origin classification model 113 may include one or more of an artificial neural network (ANN) (e.g., a vision transformer model such as a vision transformer model with shifted patch tokenization and locality self-attention), a tree-based classifier (e.g., a gradient boosted decision tree, a random forest, an extreme gradient boosted decision tree (XGBoost)), a ridge classifier, and/or the like. In some cases, the cell-of-origin classification model 113 may include a vision transformer model that is implemented with shifted patch tokenization and locality self-attention in order to increase the locality inductive bias (e.g., the assumption of a relationship between proximate pixels) of the vision transformer model and enable the vision transformer model to learn from a limited quantity of training data.

In instances where the cell-of-origin classification model 113 operates on a positron emission tomography (PET) scan without a corresponding computed tomography (CT) scan from the same timepoint, the cell-of-origin classification model 113 may include one or more tree-based classifiers (e.g., a gradient boosted decision tree, a random forest, an extreme gradient boosted decision tree (XGBoost), and/or the like). For example, in some cases, the analysis engine 110 may apply the cell-of-origin classification model 113 to determine, based at least on a tumor mask and one or more radiomic features extracted from a positron emission tomography scan, a cell-of-origin of a corresponding lesion depicted in the positron emission tomography scan. In this context, the tumor mask may include a plurality of pixels, each of which having either a first value (e.g., “1”) to indicate that the pixel is a part of a lesion or a second value (e.g., “O”) to indicate that the pixel is not a part of a lesion. The one or more radiomic features may include, for example, a size, a shape, and/or a texture of the lesion.

Alternatively and/or additionally, the one or more radiomic features may include one or more first-order statistics, a gray level co-occurrence matrix, a gray level size zone matrix, and/or a gray level run length matrix of one or more pixels of the positron emission tomography (PET) scan depicting the lesion.

In some example embodiments, the cell-of-origin classification model 113 may be trained based on a training set that includes one or more annotated training samples. For example, in some cases, the training set may be generated to include a first training sample having a first volume including a lesion depicted in a positron emission tomography (PET) scan and a first ground-truth annotation of a cell-of-origin of the lesion. Furthermore, the training set may be generated to include a second training sample having a second volume including the same lesion depicted in the positron emission tomography (PET) scan and a second ground-truth annotation of the cell-of-origin of the lesion. In some cases, the second training sample may be generated by applying one or more data augmentation techniques. For instance, in some cases, the second volume may be generated by modifying the first volume, for example, by one or more of normalizing, rotating, flipping, and changing a zoom of the first volume. Moreover, in some cases, the modifying of the first volume may be limited to one or more slices of the first volume that are within a threshold distance of a center of mass of the lesion included in the first volume.

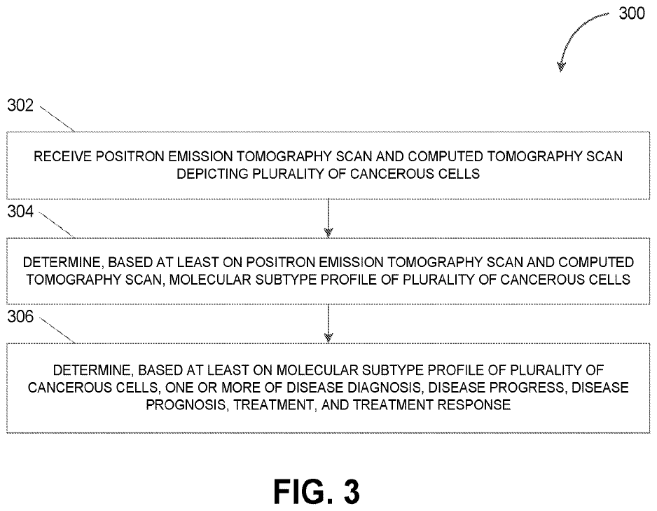

FIG. 3 depicts a flowchart illustrating an example of a process 300 for machine learning enabled cell-of-origin prediction, in accordance with some example embodiments.

Referring to FIG. 3, the process 300 may be performed by the analysis controller 110, for example, to determine the molecular subtype profile 180 of a plurality of cancerous cells depicted in a positron emission tomography (PET) scan and, in some cases, a corresponding computed tomography (CT) scan from a same timepoint.

At 302, the analysis controller 110 may receive a positron emission tomography (PET) scan and a computed tomography (CT) scan depicting a plurality of cancerous cells. For example, the analysis controller 110 may receive, from the one or more imaging devices 120, the positron emission tomography (PET) scan 210 and, in some cases, the computed tomography (CT) scan 220. In instances where the analysis controller 110 receives the positron emission tomography (PET) scan 210 as well as the computed tomography (CT) scan 220, the positron emission tomography (PET) scan 210 and the computed tomography (CT) scan 220 may be from a same timepoint. In those instances, the positron emission tomography (PET) scan 210 and the computed tomography (CT) scan 220 may be superimposed (or co-registered) such that the pixels in the positron emission tomography (PET) scan 210, whose values are indicative of a level of metabolic activity (e.g., standard uptake value (SUV)) are mapped to the pixels in the computed tomography (CT) scan 220 whose values are indicative of tissue density (or X-ray attenuation).

At 304, the analysis controller 110 may determine, based at least on the positron emission tomography (PET) scan and the computed tomography (CT) scan, a molecular subtype profile of the plurality of cancerous cells. In some example embodiments, the analysis controller 110 may apply the cell-of-origin classification model 113 to determine, for each lesion identified within the positron emission tomography (PET) scan 210 and the computed tomography (CT) scan 220, a corresponding cell-of-origin. As will be described in more detail below, the cell-of-origin classification model 113 may be applied to determine the cell-of-origin of each lesion based on a volume including the lesion extracted from the positron emission tomography (PET) scan 210 and the computed tomography (CT) scan 220. Alternatively, in some cases, the cell-of-origin classification model 113 may be applied to determine the cell-of-origin of each lesion based on one or more features of the lesion extracted from the positron emission tomography (PET) scan 210 and the computed tomography (CT) scan 220. The molecular subtype profile 180 of the plurality of cancerous cells depicted in the positron emission tomography (PET) scan 210 and the computed tomography (CT) scan 220 may be determined based on the cell-of-origin of each lesion.

At 306, the analysis controller 110 may determine, based at least on the molecular subtype profile of the plurality of cancerous cells, one or more of a disease diagnosis, a disease prognosis, a disease progress, a treatment, and a treatment response. In some example embodiments, the analysis controller 110 may determine, based at least on the molecular subtype profile 180 of the plurality of cancerous cells depicted in the positron emission tomography (PET) scan 210 and the computed tomography (CT) scan 220, one or more of a disease diagnosis, a disease prognosis, a disease progress, a treatment, and a treatment response. For example, in some cases, the plurality of cancerous cells may be associated with diffuse large B-cell lymphoma (DLBCL) and the molecular subtype profile 180 of the plurality of cancerous cells may indicate the overall cell-of-origin (e.g., germinal center B cell (GCB) or activated B cell (ABC) (or non-germinal center B cell (non-GCB)) and/or the proportion of lesions exhibiting each possible cell-of-origin. Moreover, in some cases, the analysis controller 110 may determine, based at least on the molecular subtype profile 180 of the plurality of cancerous cells, an overall survival (OS) and/or a progression-free survival (PFS) of a patient associated with the positron emission tomography (PET) scan 210 and the computed tomography (CT) scan 220 depicting the plurality of cancerous cells. Alternatively and/or additionally, the analysis controller 110 may determine, based at least on the molecular subtype profile 180 of the plurality of cancerous cells, whether the patient is suitable for a treatment (e.g., a probability of the patient being a responder (or a non-responder) for the treatment).

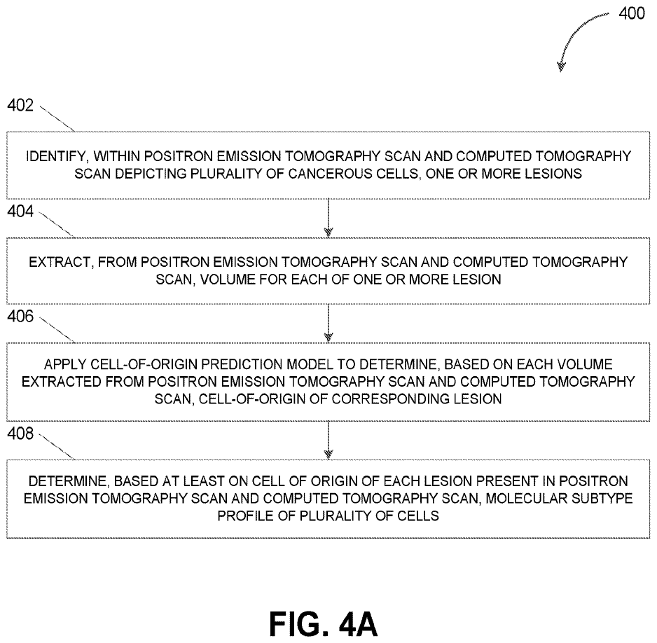

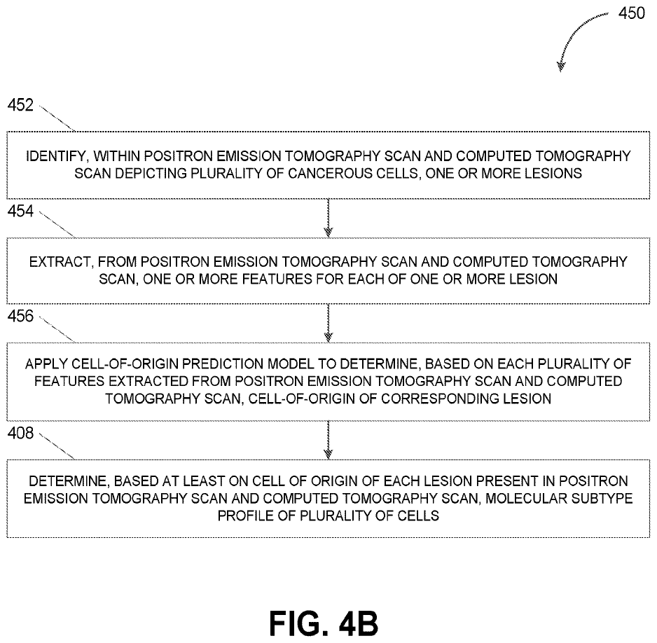

FIG. 4A depicts a flowchart illustrating an example of a process 400 for machine learning enabled cell-of-origin prediction, in accordance with some example embodiments. Referring to FIG. 4A, the process 400 may be performed by the analysis controller 110, for example, to determine the molecular subtype profile 180 of a plurality of cancerous cells depicted in a positron emission tomography (PET) scan and, in some cases, a corresponding computed tomography (CT) scan from a same timepoint. In some cases, the process 400 may implement operation 304 of the process 300.

In some cases, the analysis controller 110 may, as a part of performing the process 400, apply the cell-of-origin classification model 113, to the positron emission tomography (PET) scan 210 and, in some cases, the computed tomography (CT) scan 220, in order to determine the cell-of-origin of each lesion depicted therein. The cell-of-origin classification model 113 may be a machine learning model (e.g., a vision transformer, a tree-based classifier, a ridge classifier, and/or the like) capable of differentiating between lesions having different cells-of-origin (e.g., germinal center B cell (GCB) and activated B cell (ABC) (or non-germinal center B cell (non-GCB)) in diffuse large B-cell lymphoma (DLBCL)) based on the features (e.g., radiomic features and/or the like) present in the positron emission tomography (PET) scan 210 and, in some cases, the computed tomography (CT) scan 220.

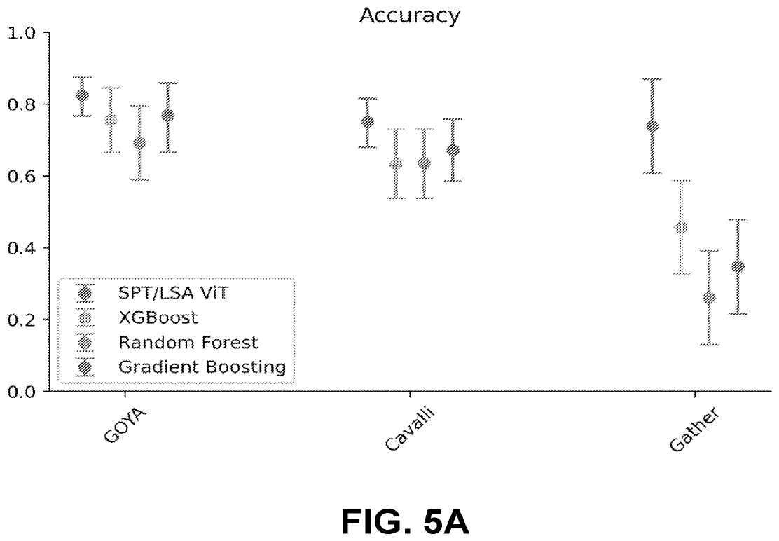

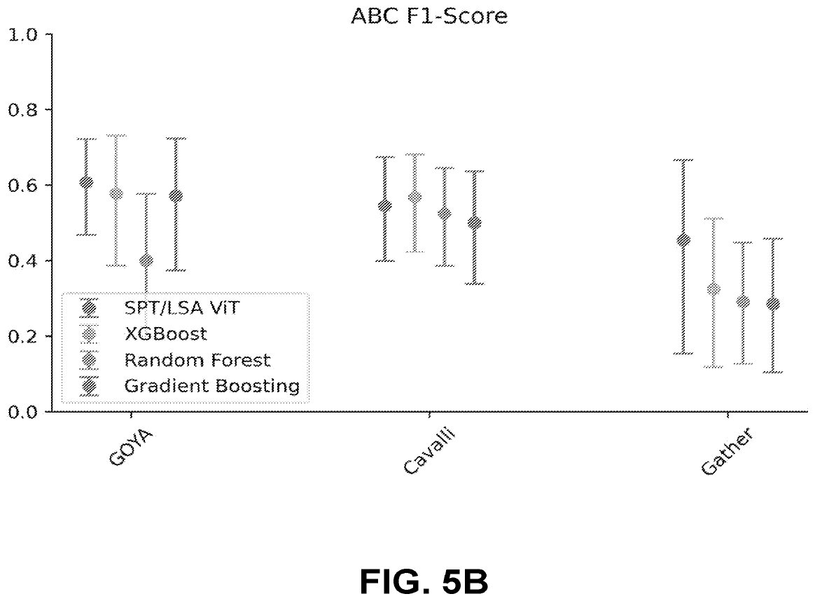

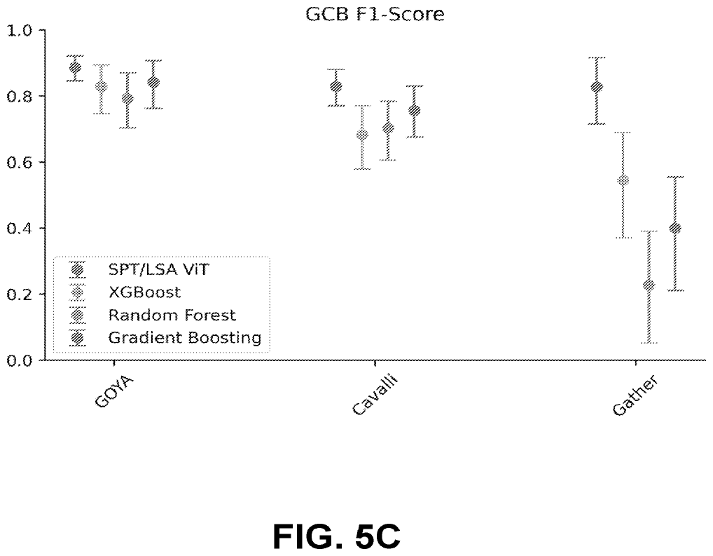

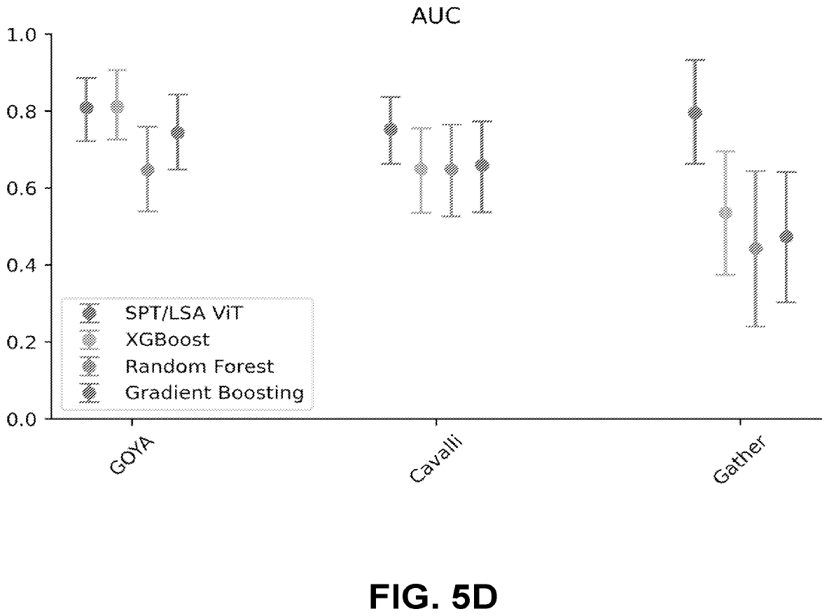

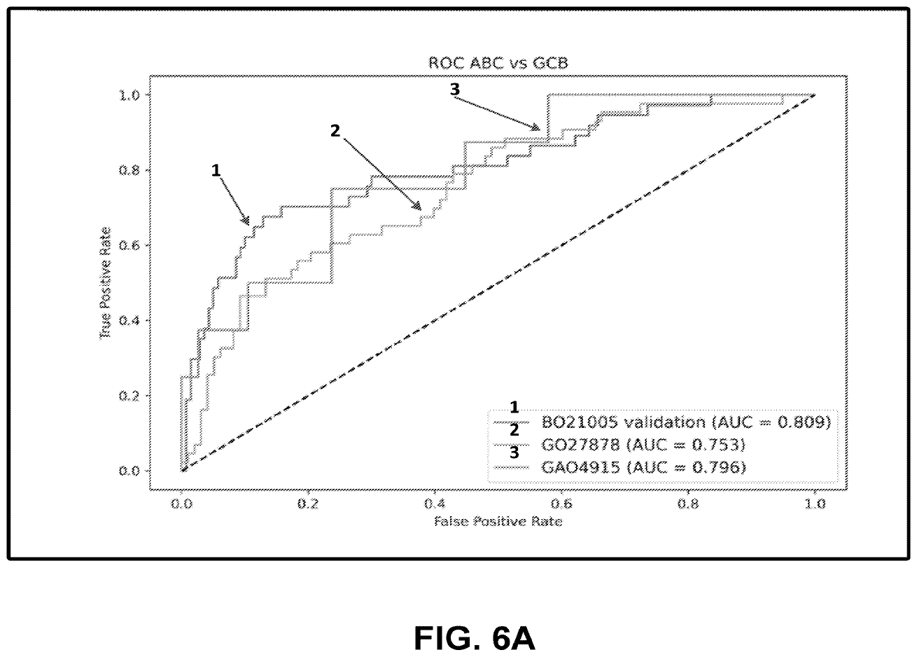

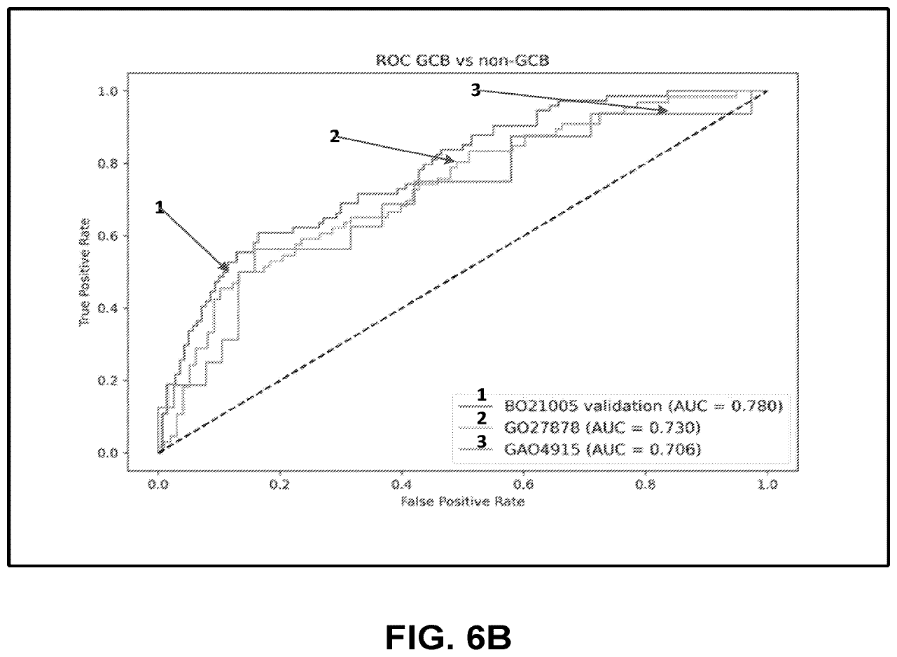

As the performance metrics shown in FIGS. 5-6 indicate, the machine learning based cell-of-origin classification model 113 applied by the analysis controller 110 when performing the process 400 is capable of making accurate differentiation between lesions having different cells-of-origin (e.g., germinal center B cell (GCB) and activated B cell (ABC) (or non-germinal center B cell (non-GCB) in diffuse large B-cell lymphoma (DLBCL)). That the cell-of-origin classification model 113 is able to determine cell-of-origin based on medical images (e.g., positron emission tomography (PET) scans and, in some cases, the corresponding computed tomography (CT) scans), which are obtained non-invasively and as a part of routine patient care, means that the process 400 may be performed to make an accurate and precise cell-of-origin determination non-invasively and with minimal added resources. Contrastingly, conventional techniques for determining cell-of-origin (COO) require invasive assays to extract tumor tissue and data analytics with either limited availability or limited performance, limited reproducibility, and high inter-reader variability.

Furthermore, the analysis controller 110 may perform the process 400 to generate the molecular subtype profile 180 of the plurality of cancerous cells based on data associated with individual lesions. In cases where there are multiple lesions present in the positron emission tomography (PET) scan 210 and the computed tomography (CT) scan 220, the molecular subtype profile 180 may account for the different cells-of-origin that may be present in different lesions. Contrastingly, the aforementioned conventional techniques rely on data associated with a single tumor sample, meaning that conventional techniques are unable to capture valuable biological insights, such as the heterogeneity and/or uniformity of cells-of-origin across different lesions.

At 402, the analysis controller 110 may identify, within a positron emission tomography (PET) scan and a computed tomography (CT) scan depicting a plurality of cancerous cells, one or more lesions. In some example embodiments, the analysis controller 110 may segment the positron emission tomography (PET) scan 210 and, in some cases, the computed tomography (CT) scan 220, to identify the one or more lesions depicted therein. For example, in some cases, the analysis controller 110 may identify the one or more lesions by applying the segmentation model 112 (e.g., an artificial neural network and/or the like) trained to assign, to each pixel in the positron emission tomography (PET) scan 210 and/or the computed tomography (CT) scan 220, a label having a first value to indicate the pixel depicts a lesion and a second value to indicate the pixel does not depict a lesion. In some cases, a pixel in the positron emission tomography (PET) scan 210 and a corresponding pixel in the co-registered computed tomography (CT) scan 220 may be identified as depicting a lesion if the values of these pixels satisfy one or more corresponding thresholds (e.g., for level of metabolic activity, tissue density, and/or X-ray attenuation). Alternatively and/or additionally, the analysis controller 110 may identify the one or more lesions by first applying thresholding to identify one or more objects present in the positron emission tomography (PET) scan 210 and the corresponding computed tomography (CT) scan 220 before applying one or more machine learning models (e.g., logistic regression models, tree-based classifiers, fully-connected neural networks, and/or the like) trained to perform object classification.

At 404, the analysis controller 110 may extract, from the positron emission tomography (PET) scan and the computed tomography (CT) scan, a volume for each of the one or more lesions. In some example embodiments, the analysis controller 110 may extract, from the positron emission tomography (PET) scan 210 and, in some cases, the computed tomography (CT) scan 220, the first volume 230a including the first lesion and the second volume 230b including the second lesion. Each of the first volume 230a and the second volume 230b may be a three-dimensional volume having a plurality of two-dimensional patches (e.g., axial patches, coronal patches, and/or the like). In some cases, to extract the first volume 230a including the first lesion, the analysis controller 110 may extract a first plurality of two dimensional patches centered around a first center of mass of the first lesion. Meanwhile, to extract the second volume 230b, the analysis controller 110 may extract a second plurality of two-dimensional patches centered around a second center of mass of the second lesion.

At 406, the analysis controller 110 may apply the cell-of-origin classification model 113 to determine, based on each volume extracted from the positron emission tomography (PET) scan and the computed tomography (CT) scan, a cell-of-origin of a corresponding lesion. In some example embodiments, the cell-of-origin classification model 113 may be trained to determine the cell-of-origin of a lesion based at least on a three-dimensional volume containing the lesion from the positron emission tomography (PET) scan 210 and, in some cases, a corresponding three-dimensional volume containing the lesion from the co-registered computed tomography (CT) scan 220. The intensity values of the pixels within the three-dimensional volume extracted from the positron emission tomography (PET) scan 210 may correspond to a level of cellular metabolic activity (e.g., standard uptake value (SUV) corresponding to the quantity of glucose intake) whereas the intensity values of the pixels within the three-dimensional volume extracted from the computed tomography (CT) scan 220 may correspond to tissue density or X-ray attenuation. Accordingly, in some cases, the cell-of-origin classification model 113 may be trained to determine the cell-of-origin of the lesion based on the level of metabolic activity (e.g., standard uptake value (SUV)), the tissue density, and/or the X-ray attenuation exhibited by the lesion and its surrounding environment. For instance, the analysis controller 110 may apply the cell-of-origin classification model 113 to determine, based at least on the first volume 230a, the first cell-of-origin 240a of the first lesion included in the first volume 230a. Furthermore, the analysis controller 110 may apply the cell-of origin classification model 113 to determine, based at least on the second volume 230b, the second cell-of-origin 240b of the second lesion included in the second volume 230b.

At 408, the analysis controller 110 may determine, based at least on the cell of origin of each lesion present in the positron emission tomography (PET) scan and the computed tomography (CT) scan, a molecular subtype profile for the plurality of cancerous cells. In some example embodiments, the analysis controller 110 may determine the molecular subtype profile 180 of the plurality of cancerous cells depicted in the positron emission tomography (PET) scan 210 and, in some cases, the computed tomography (CT) scan 220, based on the first cell-of-origin 240a of the first lesion included in the first volume 230a and the second cell-of-origin 240b of the second lesion included in the second volume 230b. For example, in some cases, the analysis controller 110 may determine, for inclusion in the molecular subtype profile 180 of the plurality of cancerous cells to include, an overall cell-of-origin based at least on the first cell-of-origin 240a of the first lesion and the second cell-of-origin 240b of the second lesion. The overall cell-of-origin of the plurality of cancerous cells may, in some cases, be identified as being a particular cell-of-origin (e.g., germinal center B cell (GCB) or activated B cell (ABC) (or non-germinal center B-cell (non-GCB)) if the probability of a threshold quantity (e.g., percentage, ratio, and/or the like) of lesions being associated with that particular cell-of-origin satisfies one or more thresholds. In some cases, the analysis engine 110 may determine the overall cell-of-origin of the plurality of cancerous cells by at least applying a machine learning model (e.g., a neural network such as a recurrent neural network (RNN) and/or the like) that operates on an embedding sequence that includes, for each possible cell-of-origin and each lesion, a probability that the lesion exhibits the corresponding cell-of-origin. Alternatively and/or additionally, the analysis controller 110 may determine, for inclusion in the molecular subtype profile 180, one or more metrics indicative of a heterogeneity and/or uniformity between the first cell-of-origin 240a of the first lesion and the second cell-of-origin 240b of the second lesion.

FIG. 4B depicts a flowchart illustrating another example of a process 450 for machine learning enabled cell-of-origin prediction, in accordance with some example embodiments. Referring to FIG. 4B, the process 450 may be performed by the analysis controller 110, for example, to generate the molecular subtype profile 180 of a plurality of cancerous cells depicted in a positron emission tomography (PET) scan and, in some cases, a corresponding computed tomography (CT) scan from a same timepoint. In some cases, the process 400 may implement operation 304 of the process 300.

In some cases, the analysis controller 110 may, as a part of performing the process 450, apply the cell-of-origin classification model 113, to the positron emission tomography (PET) scan 210 and, in some cases, the computed tomography (CT) scan 220, in order to determine the cell-of-origin of each lesion depicted therein. As the performance metrics shown in FIGS. 5-6 indicate, the machine learning based cell-of-origin classification model 113 applied by the analysis controller 110 when performing the process 450 is capable of making accurate differentiation between lesions having different cells-of-origin (e.g., germinal center B cell (GCB) and activated B cell (ABC) (or non-germinal center B cell (non-GCB) in diffuse large B-cell lymphoma (DLBCL)). That the cell-of-origin classification model 113 is able to determine cell-of-origin based on medical images (e.g., positron emission tomography (PET) scans and, in some cases, the corresponding computed tomography (CT) scans), which are obtained non-invasively and as a part of routine patient care, means that the process 450 may be performed to make an accurate and precise cell-of-origin determination non-invasively and with minimal added resources. Contrastingly, conventional techniques for determining cell-of-origin (COO) require invasive assays to extract tumor tissue and data analytics with either limited availability or limited performance, limited reproducibility, and high inter-reader variability. Moreover, conventional techniques rely on data associated with a single tumor sample whereas the analysis controller 110 may perform the process 450 to generate the molecular subtype profile 180 to account for the different cells-of-origin that may be present in different lesions. Thus, the molecular subtype profile 180 generated by the analysis controller 110 performing the process 450 may capture valuable biological insights, such as the heterogeneity and/or uniformity of cells-of-origin across different lesions, that eludes conventional techniques.

At 452, the analysis controller 110 may identify, within a positron emission tomography (PET) scan and a computed tomography (CT) scan depicting a plurality of cancerous cells, one or more lesions. As noted, in some example embodiments, the analysis controller 110 may segment the positron emission tomography (PET) scan 210 and, in some cases, the computed tomography (CT) scan 220, to identify the one or more lesions depicted therein. In some cases, the analysis controller 110 may identify the one or more lesions by applying the segmentation model 112 (e.g., an artificial neural network and/or the like) or a machine learning model (e.g., logistic regression models, tree-based classifiers, fully-connected neural networks, and/or the like) trained to classify one or more objects identified (e.g., through thresholding of pixel values) in the positron emission tomography (PET) scan 210 and/or the computed tomography (CT) scan 220.

At 454, the analysis controller 110 may extract, from the positron emission tomography (PET) scan and the computed tomography (CT) scan, a plurality of features for each of the one or more lesions. The cell-of-origin of a lesion may be determined based on one or more features present in the positron emission tomography (PET) scan 210 and, in some cases, the computed tomography (CT) scan 220 depicting the lesion. Accordingly, in some example embodiments, the analysis controller 110 may extract, from the positron emission tomography (PET) scan 210 and, in some cases, the computed tomography (CT) scan 220, the first plurality of features 235a of the first lesion and the second plurality of features 235b of the second lesion. The first plurality of features 235a and the second plurality of features 235b may include a variety of radiomic features including, for example, a size, a shape, and/or a texture of the corresponding lesion. Other examples of radiomic features include one or more first-order statistics such as a range, a maximum, a minimum, a median, a mode, and/or a mean pixel value of the one or more pixels depicting the lesion in the positron emission tomography (PET) scan 210 and, in some cases, the computed tomography (CT) scan 220. In some cases, the first plurality of features 235a and the second plurality of features 235b may further include other radiomic features such as a gray level co-occurrence matrix, a gray level size zone matrix, and/or a gray level run length matrix of the one or more pixels depicting the corresponding lesion in the positron emission tomography (PET) scan 210 and, in some cases, the computed tomography (CT) scan 220.

At 456, the analysis controller 110 may apply the cell-of-origin classification model 113 to determine, based on each plurality of features extracted from the positron emission tomography (PET) scan and the computed tomography (CT) scan, a cell-of-origin of a corresponding lesion. For example, in some cases, the cell-of-origin classification model 113 may be applied to determine, based at least on the first plurality of features 235a, the first cell-of-origin 240a of the first lesion. Furthermore, the cell-of-origin classification model 113 may be applied to determine, based at least on the second plurality of features 235b, the second cell-of-origin 240b of the second lesion. As noted, the cell-of-origin classification model 113 may include one or more of an artificial neural network (ANN) (e.g., a vision transformer model such as a vision transformer model with shifted patch tokenization and locality self-attention), a tree-based classifier (e.g., a gradient boosted decision tree, a random forest, an extreme gradient boosted decision tree (XGBoost)), a ridge classifier, and/or the like. In the example of the process 450, the cell-of-origin classification model 113 may be trained to learn the nexus between the cell-of-origin of a lesion and the various radiomic features of the lesion present in a positron emission tomography (PET) scan and/or a computed tomography (CT) scan depicting the lesion.

At 458, the analysis controller 110 may determine, based at least on the cell of origin of each lesion present in the positron emission tomography (PET) scan and the computed tomography (CT) scan, a molecular subtype profile of the plurality of cancerous cells. In some example embodiments, the analysis controller 110 may determine, for inclusion in the molecular subtype profile 180 of the plurality of cancerous cells depicted in the positron emission tomography (PET) scan 210 and, in some cases, the computed tomography (CT) scan 220, an overall cell-of-origin determined based at least on the first cell-of-origin 240a of the first lesion included in the first volume 230a and the second cell-of-origin 240b of the second lesion included in the second volume 230b. As noted, in some cases, the overall cell-of-origin may include, for each possible cell-of-origin, a corresponding probability determined based on the probabilities of each individual lesion having that cell-of-origin (e.g., a maximum, a minimum, a mean, a median, and/or a mode of the first probability of the first lesion and the second probability of the second lesion having the cell-of-origin). In some cases, the analysis engine 110 may determine the overall cell-of-origin by at least applying a machine learning model (e.g., a neural network such as a recurrent neural network (RNN) and/or the like) that operates on an embedding sequence that includes, for each possible cell-of-origin and each lesion, a corresponding probability that the lesion exhibits the cell-of-origin. Alternatively and/or additionally, the molecular subtype profile 180 of the plurality of cancerous cells may be generated to include one or more metrics indicative of a heterogeneity and/or uniformity of the cells-of-origin present across the different lesions (e.g., between the first cell-of-origin 240a of the first lesion and the second cell-of-origin 240b of the second lesion).

Table 1 below depicts various performance metrics (e.g., sensitivity, specificity, and area under the curve (AUC)) for differentiation between the germinal center B cell (GCB) and activated B cell (ABC) molecular subtypes of different implementations of the cell-of-origin classification model 113 (e.g., gradient boosted decision tree (GB), random forest (RF), extreme gradient boosted decision tree (XGBoost), and vision transformer model with shifted patch tokenization and locality self-attention (SPT/LSA ViT)) across various clinical datasets (e.g., GOYA holdout, Cavalli, and Gather).

TABLE 1

| | | | |
|---|---|---|---|
| N | Sensitivity | Specificity | AUC |

GOYA Holdout

| | | | | |
|---|---|---|---|---|
| GB | 177 | 81 | 63 | 0.744 |
| RF | | 78 | 42 | 0.648 |
| XGBoost | | 78 | 68 | 0.811 |
| SPT/LSA ViT | | 87 | 65 | 0.809 |

Cavalli

| | | | | |
|---|---|---|---|---|
| GB | 141 | 74 | 53 | 0.659 |
| RF | | 62 | 66 | 0.649 |
| XGBoost | | 72 | 38 | 0.650 |
| SPT/LSA ViT | | 87 | 49 | 0.753 |

Gather

| | | | | |
|---|---|---|---|---|
| GB | 46 | 74 | 53 | 0.659 |
| RF | | 62 | 66 | 0.649 |
| XGBoost | | 72 | 38 | 0.650 |
| SPT/LSA ViT | | 87 | 49 | 0.753 |

Table 2 below depicts various performance metrics (e.g., sensitivity, specificity, and area under the curve (AUC)) for differentiation between the germinal center B cell (GCB) and non-germinal center B cell (non-GCB) molecular subtypes of different implementations of the cell-of-origin classification model 113 (e.g., gradient boosted decision tree (GB), random forest (RF), extreme gradient boosted decision tree (XGBoost), and vision transformer model with shifted patch tokenization and locality self-attention (SPT/LSA ViT)) across various clinical datasets (e.g., GOYA holdout, Cavalli, and Gather).

TABLE 2

| | | | |
|---|---|---|---|
| N | Sensitivity | Specificity | AUC |

GOYA Holdout

| | | | | |
|---|---|---|---|---|
| GB | 214 | 81 | 43 | 0.593 |
| RF | | 46 | 70 | 0.581 |
| XGBoost | | 69 | 67 | 0.693 |
| SPT/LSA ViT | | 84 | 59 | 0.780 |

Cavalli

| | | | | |
|---|---|---|---|---|
| GB | 164 | 74 | 45 | 0.608 |
| RF | | 38 | 82 | 0.604 |
| XGBoost | | 57 | 71 | 0.634 |
| SPT/LSA ViT | | 87 | 48 | 0.730 |

Gather

| | | | | |
|---|---|---|---|---|
| GB | 54 | 34 | 75 | 0.502 |
| RF | | 61 | 50 | 0.533 |
| XGBoost | | 68 | 31 | 0.581 |
| SPT/LSA ViT | | 84 | 50 | 0.706 |

FIG. 5A depicts a graph illustrating the accuracy of different implementations of the cell-of-origin classification model 113 (e.g., SPT/LSA VIT, extreme gradient boosted decision tree (XG Boost), random forest (RF), and gradient boosted decision tree (GB) from left to right) across different clinical datasets (e.g., GOYA holdout, Cavalli, and Gather). The F1-scores of each implementation of the cell-of-origin classification model 113 (e.g., SPT/LSA VIT, extreme gradient boosted decision tree (XG Boost), random forest (RF), and gradient boosted decision tree (GB) from left to right) for the activated B cell (ABC) molecular subtype across the different clinical datasets (e.g., GOYA holdout, Cavalli, and Gather) are shown in FIG. 5B. The F1-scores of each implementation of the cell-of-origin classification model 113 (e.g., SPT/LSA VIT, extreme gradient boosted decision tree (XG Boost), random forest (RF), and gradient boosted decision tree (GB) from left to right) for the germinal center B cell (GCB) molecular subtype across the different clinical datasets (e.g., GOYA holdout, Cavalli, and Gather) are shown in FIG. 5C. FIG. 5D depicts a graph illustrating the area under the curve (AUC) achieved by the different implementations of the cell-of-origin classification model 113 (e.g., SPT/LSA ViT, extreme gradient boosted decision tree (XG Boost), random forest (RF), and gradient boosted decision tree (GB) from left to right) across different clinical datasets (e.g., GOYA holdout, Cavalli, and Gather).

FIG. 6A depicts a graph illustrating the receiver operating characteristic (ROC) curves of the cell-of-origin classification model 113 in differentiating between the activated B cell (ABC) molecular subtype and the germinal center B cell (GCB) molecular subtype across different test sets. FIG. 6B depicts a graph illustrating the receiver operating characteristic (ROC) curves of the cell-of-origin classification model 113 in differentiating between the germinal center B cell (GCB) molecular subtype and the non-germinal center B cell (non-GCB) molecular subtype across different test sets.

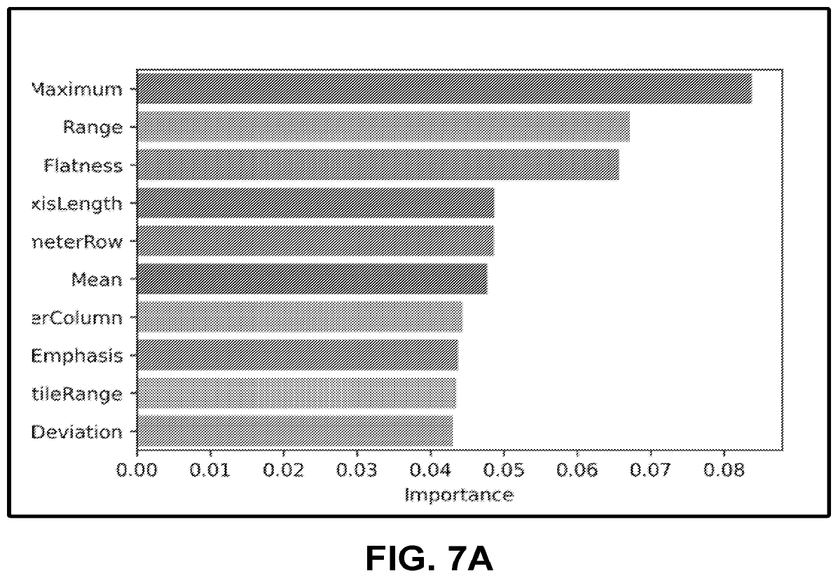

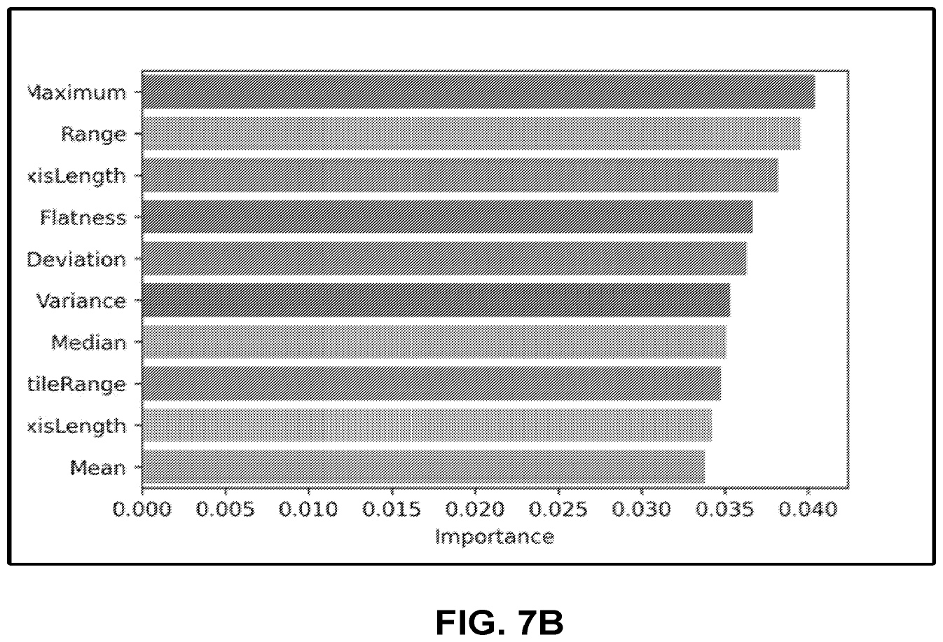

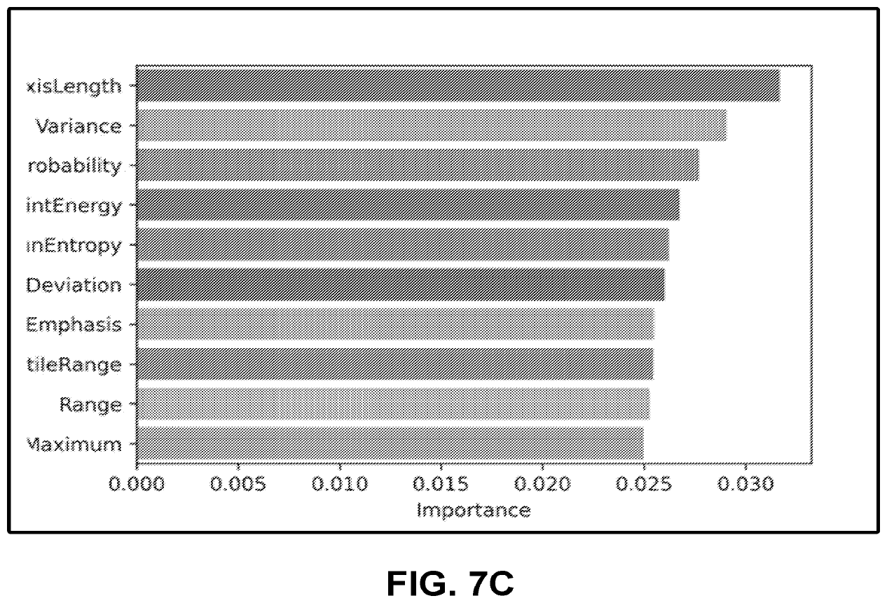

FIG. 7A depicts a graph illustrating the importance of various radiomic features for the cell-of-origin classification model 113 implemented using a gradient boosted decision tree. FIG. 7B depicts a graph illustrating the importance of various radiomic features for the cell-of-origin classification model 113 implemented using a random forest (RF). FIG. 7C depicts a graph illustrating the importance of various radiomic features for the cell-of-origin classification model 113 implemented using an extreme gradient boosted decision tree (XGBoost).

Table 3 below depicts various performance metrics (e.g., sensitivity, specificity, and area under the curve (AUC)) for differentiation between the germinal center B cell (GCB) and activated B cell (ABC) molecular subtypes of different implementations of the cell-of-origin classification model 113 (e.g., ridge classifier and a fully connected convolutional neural network (FCCC)) across various clinical datasets (e.g., GOYA holdout, Cavalli, and Gather).

TABLE 3

| | | | |
|---|---|---|---|
| N | Sensitivity | Specificity | AUC |

GOYA Holdout

| | | | | |
|---|---|---|---|---|
| Ridge | 177 | 25 | 89 | 0.611 |
| FCNN | | 90 | 25 | 0.625 |

Cavalli

| | | | | |
|---|---|---|---|---|
| Ridge | 141 | 32 | 84 | 0.653 |
| FCNN | | 89 | 21 | 0.520 |

Gather

| | | | | |
|---|---|---|---|---|
| Ridge | 46 | 53 | 62 | 0.618 |
| FCNN | | 91 | 28 | 0.550 |

In view of the above-described implementations of subject matter this application discloses the following list of examples, wherein one feature of an example in isolation or more than one feature of said example taken in combination and, optionally, in combination with one or more features of one or more further examples are further examples also falling within the disclosure this application.
Item 1: A computer-implemented method, comprising receiving a first positron emission tomography (PET) scan depicting a plurality of cancerous cells, identifying a first lesion depicted in the first PET scan, applying a cell-of-origin classification model to determine, based at least on the first lesion depicted in the first PET scan, a first cell-of-origin associated with the first lesion, and determining, based at least on the first cell-of-origin of the first lesion, a molecular subtype profile for the plurality of cancerous cells depicted in the first PET scan.
Item 2: The method of Item 1, wherein the first cell-of-origin of the first lesion includes, for each possible cell-of-origin, a probability that one or more cancerous cells forming the first lesion is of that cell-of-origin.
Item 3: The method of Item 1 or Item 2, further comprising identifying a second lesion depicted in the first PET scan, applying the cell-of-origin classification model to determine, based at least on the second lesion depicted in the first PET scan, a second cell-of-origin associated with the second lesion, and determining, further based on the second cell-of-origin of the second lesion, the molecular subtype profile for the plurality of cancerous cells depicted in the first PET scan.
Item 4: The method of Item 3, wherein the molecular subtype profile of the plurality of cancerous cells depicted in the first PET scan includes, for each possible cell-of-origin, a probability that an overall cell-of-origin of the plurality of cancerous cells is that cell-of-origin.
Item 5: The method of Item 4, wherein the probability of the overall cell-of-origin of the plurality of cancerous cells being a particular cell-of-origin is a maximum, a minimum, a mean, a median, and/or a mode of a respective probability of each of the first lesion and the second lesion having that particular cell-of-origin.
Item 6: The method according to any one of Items 3 to 4, wherein the molecular subtype profile of the plurality of cancerous cells depicted in the first PET scan includes, for each possible cell-of-origin, a corresponding proportion of lesions having that cell-of-origin.
Item 7: The method according to any one of Items 3 to 5, wherein the molecular subtype profile of the plurality of cancerous cells depicted in the first PET scan is determined by at least generating a embedding sequence to include the first cell-of-origin of the first lesion and the second cell-of-origin of the second lesion, and applying a machine learning model to determine, based at least on the embedding sequence, an overall cell-of-origin of the plurality of cancerous cells in the first PET scan.
Item 8: The method of Item 7, wherein the machine learning model is a recurrent neural network.
Item 9: The method according to any one of Items 1 to 8, further comprising extracting, from the first PET scan, a volume including the first lesion identified in the first PET scan, and applying the cell-of-origin classification model to determine, based at least on the volume extracted from the first PET scan, the first cell-of-origin associated with the first lesion.
Item 10: The method of Item 9, wherein the volume including the first lesion is extracted by at least determining, within the first PET scan, a center of mass of the first lesion, and extracting, based at least on the center of mass of the first lesion, the volume.
Item 11: The method of Item 10, wherein the volume is a three-dimensional volume comprising a plurality of two-dimensional patches centered around the center of mass of the first lesion.
Item 12: The method of Item 11, wherein the plurality of two-dimensional patches include a plurality of axial patches or a plurality of coronal patches.
Item 13: The method according to any one of Items 1 to 12, wherein the first lesion is identified by at least applying a segmentation model to identify, within the first PET scan, a plurality of pixels corresponding to the first lesion.
Item 14: The method according to any one of Items 1 to 13, further comprising: extracting, from the first PET scan, a plurality of features associated with the first lesion identified in the first PET scan, and applying the cell-of-origin classification model to determine, based at least on the plurality of features extracted from the first PET scan, the first cell-of-origin associated with the first lesion.
Item 15: The method of Item 14, wherein the plurality of features include a size of the first lesion, a shape of the first lesion, and/or a texture of the first lesion.
Item 16: The method according to any one of Items 14 to 15, wherein the plurality of features include one or more first-order statistics associated with one or more pixels depicting the first lesion in the first PET scan.
Item 17: The method according to any one of Items 14 to 16, wherein the plurality of features include a gray level co-occurrence matrix, a gray level size zone matrix, and/or a gray level run length matrix of one or more pixels depicting the first lesion in the first PET scan.
Item 18: The method according to any one of Items 1 to 17, further comprising receiving a computed tomography (CT) scan from a same timepoint as the first PET scan, identifying the first lesion depicted in the CT scan, and applying the cell-of-origin classification model to determine, based on the first lesion depicted in the first PET scan and the CT scan, the first cell-of-origin associated with the first lesion.
Item 19: The method of Item 18, wherein each pixel included in the CT scan is associated with an intensity value corresponding to a tissue density or an x-ray attenuation.
Item 20: The method of Item 18, wherein each pixel in the first PET scan is associated with an intensity value corresponding to a level of metabolic activity.
Item 21: The method of Item 18, further comprising determining, based on at least one of the CT scan and the first PET scan, a tumor mask corresponding to the first lesion, and applying the cell-of-origin classification model to determine, further based at least on the tumor mask, the cell-of-origin of the first lesion.
Item 22: The method of Item 21, further comprising determining, based on at least one of the CT scan and the first PET scan, an organ mask corresponding to one or more organs depicted in the CT scan and the first PET scan, and applying the cell-of-origin classification model to determine, further based at least on the organ mask, the cell-of-origin of the first lesion.
Item 23: The method according to any one of Items 1 to 22, further comprising receiving a second positron emission tomography (PET) scan from a different timepoint as the first PET scan, identifying the first lesion depicted in the second PET scan, and applying the cell-of-origin classification model to determine, based on the first lesion depicted in the first PET scan and the second PET scan, the first cell-of-origin of the first lesion.
Item 24: The method according to any one of Items 1 to 23, further comprising receiving a first computed tomography (CT) scan from a same timepoint as the first PET scan and a second CT scan from a same timepoint as the second PET scan, identifying the first lesion depicted in the first CT scan and the first lesion depicted in the second CT scan, and applying the cell-of-origin classification model to determine, based at least on the first lesion depicted in the first PET scan, the first CT scan, the second PET scan, and the second CT scan, the first cell-of-origin of the first lesion.
Item 25: The method according to any one of Items 1 to 24, further comprising: determining, based at least on the molecular subtype profile of the plurality of cancerous cells depicted in the first PET scan, a disease diagnosis, a disease prognosis, a disease progress, a treatment, and/or a treatment response for a patient associated with the first PET scan.
Item 26: The method according to any one of Items 1 to 25, wherein the plurality of cancerous cells are associated with diffuse large B-cell lymphoma (DLBCL).
Item 27: The method according to any one of Items 1 to 26, wherein the cell-of-origin classification model is trained to differentiate between a plurality of cells-of-origin.
Item 28: The method of Item 27, wherein the plurality of cells-of-origin include germinal center B cell (GCB) and activated B cell (ABC).
Item 29: The method of Item 27, wherein the plurality of cells-of-origin include germinal center B cell (GCB) or non-germinal center B cell (non-GCB).
Item 30: The method according to any one of Items 1 to 29, wherein the cell-of-origin classification model includes an artificial neural network (ANN).
Item 31: The method according to any one of Items 1 to 30, wherein the cell-of-origin classification model includes a vision transformer.
Item 32: The method according to any one of Items 1 to 31, wherein the cell-of-origin classification model includes a vision transformer with shifted patch tokenization and locality self-attention.
Item 33: The method according to any one of Items 1 to 32, wherein the cell-of-origin classification model includes a tree-based classifier.
Item 34: The method according to any one of Items 1 to 33, wherein the cell-of-origin classification model includes a ridge classifier.
Item 35: The method according to any one of Items 1 to 34, further comprising training, based at least on a training set, the cell-of-origin classification model to determine, based on at least a portion of a positron emission tomography (PET) scan, a cell-of-origin of at least one lesion depicted in the PET scan.
Item 36: The method of Item 35, further comprising extracting, from a second positron emission tomography (PET) scan, a first volume including a second lesion depicted in the second PET scan, generating a first training sample to include the first volume and a first ground-truth annotation of a second cell-of-origin of the second lesion, and generating the training set to include the first training sample.
Item 37: The method of Item 36, further comprising generating, based at least on the first volume, a second volume, and generating, for inclusion in the training set, a second training sample including the second volume and the first ground-truth annotation of the second cell-of-origin of the second lesion.
Item 38: The method of Item 37, wherein the second volume is generated by modifying the first volume.
Item 39: The method according to any one of Items 37 to 38, wherein the modifying includes one or more of normalizing, rotating, flipping, and changing a zoom of the first volume.
Item 40: The method according to any one of Items 37 to 39, wherein the modifying of the first volume includes modifying one or more slices of the first volume that are within a threshold distance of a center of mass of the second lesion.
Item 40: A system, comprising at least one data processor, and at least one memory storing instructions, which when executed by the at least one data processor, result in operations comprising the method of any of Items 1 to 40.
Item 41: A non-transitory computer readable medium storing instructions, which when executed by at least one data processor, result in operations comprising the method of any of Items 1 to 40.

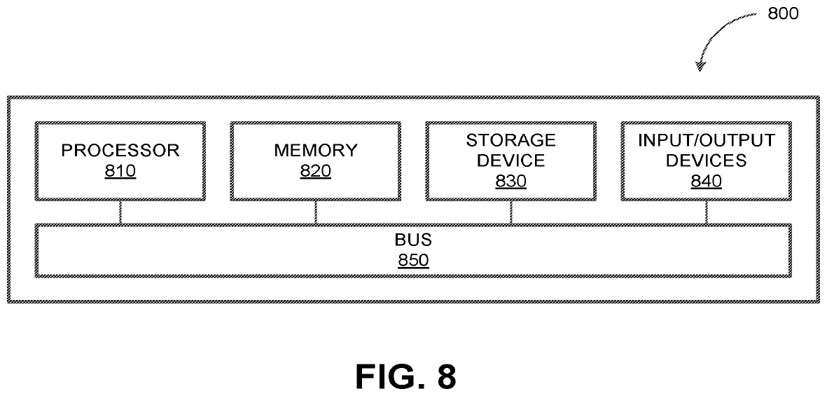

FIG. 8 depicts a block diagram illustrating an example of a computing system 800 consistent with implementations of the current subject matter. Referring to FIGS. 1-8, the computing system 800 can be used to implement the analysis controller 110, the one or more imaging devices 120, the client device 130, and/or any components therein.

As shown in FIG. 8, the computing system 800 can include a processor 810, a memory 820, a storage device 830, and an input/output device 840. The processor 810, the memory 820, the storage device 830, and the input/output device 840 can be interconnected via a system bus 850. The processor 810 is capable of processing instructions for execution within the computing system 800. Such executed instructions can implement one or more components of, for example, the analysis controller 110, the one or more imaging devices 120, and the client device 130. In some example embodiments, the processor 810 can be a single-threaded processor. Alternately, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a solid state drive, a floppy disk device, a hard disk device, an optical disk device, or a tape device, or other suitable persistent storage means. The input/output device 840 provides input/output operations for the computing system 800. In some example embodiments, the input/output device 840 includes a keyboard and/or pointing device. In various implementations, the input/output device 840 includes a display unit for displaying graphical user interfaces.

According to some example embodiments, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example embodiments, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of’ or “one or more of’ may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B:” “one or more of A and B:” and “A and/or B” are each intended to mean “A alone, B alone, or A and B together.” A similar interpretation is also intended for lists including three or more items. For example, the phrases “at least one of A, B, and C:” “one or more of A, B, and C:” and “A, B, and/or C” are each intended to mean “A alone, B alone, C alone, A and B together, A and C together, Band C together, or A and Band C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results.

Other implementations may be within the scope of the following claims.

1. A system, comprising:
at least one data processor; and
at least one memory storing instructions, which when executed by the at least one data processor, result in operations comprising:
receiving a first positron emission tomography (PET) scan depicting a plurality of cancerous cells;
identifying a first lesion depicted in the first PET scan;
applying a cell-of-origin classification model to determine, based at least on the first lesion depicted in the first PET scan, a first cell-of-origin associated with the first lesion; and
determining, based at least on the first cell-of-origin of the first lesion, a molecular subtype profile for the plurality of cancerous cells depicted in the first PET scan.

2. The system of claim 1, wherein the first cell-of-origin of the first lesion includes, for each possible cell-of-origin, a probability that one or more cancerous cells forming the first lesion is of that cell-of-origin.

3. The system of claim 1, further comprising:
identifying a second lesion depicted in the first PET scan;
applying the cell-of-origin classification model to determine, based at least on the second lesion depicted in the first PET scan, a second cell-of-origin associated with the second lesion; and
determining, further based on the second cell-of-origin of the second lesion, the molecular subtype profile for the plurality of cancerous cells depicted in the first PET scan,
wherein the molecular subtype profile of the plurality of cancerous cells depicted in the first PET scan includes, for each possible cell-of-origin, a probability that an overall cell-of-origin of the plurality of cancerous cells is that cell-of-origin,
wherein the probability of the overall cell-of-origin of the plurality of cancerous cells being a particular cell-of-origin is a maximum, a minimum, a mean, a median, and/or a mode of a respective probability of each of the first lesion and the second lesion having that particular cell-of-origin, and
wherein the molecular subtype profile of the plurality of cancerous cells depicted in the first PET scan includes, for each possible cell-of-origin, a corresponding proportion of lesions having that cell-of-origin.

4. (canceled)

5. (canceled)

6. (canceled)

7. The system of claim 3, wherein the molecular subtype profile of the plurality of cancerous cells depicted in the first PET scan is determined by at least
generating a embedding sequence to include the first cell-of-origin of the first lesion and the second cell-of-origin of the second lesion, and
applying a machine learning model to determine, based at least on the embedding sequence, an overall cell-of-origin of the plurality of cancerous cells in the first PET scan.

8. (canceled)

9. The system of claim 1, further comprising:
extracting, from the first PET scan, a volume including the first lesion identified in the first PET scan, wherein the volume including the first lesion is extracted by at least determining, within the first PET scan, a center of mass of the first lesion, and extracting, based at least on the center of mass of the first lesion, the volume, wherein the volume is a three-dimensional volume comprising a plurality of two-dimensional patches centered around the center of mass of the first lesion, and wherein the plurality of two-dimensional patches include a plurality of axial patches or a plurality of coronal patches; and
applying the cell-of-origin classification model to determine, based at least on the volume extracted from the first PET scan, the first cell-of-origin associated with the first lesion.

10. (canceled)

11. (canceled)

12. (canceled)

13. The system of claim 1, wherein the first lesion is identified by at least applying a segmentation model to identify, within the first PET scan, a plurality of pixels depicting the first lesion.

14. The system of claim 1, further comprising:
extracting, from the first PET scan, a plurality of features associated with the first lesion identified in the first PET scan, wherein the plurality of features include one or more of
a size of the first lesion, a shape of the first lesion, a texture of the first lesion, a first-order statistic associated with one or more pixels depicting the first lesion in the first PET scan, a gray level co-occurrence matrix of the one or more pixels, a gray level size zone matrix of the one or more pixels, and/or a gray level run length matrix of the one or more pixels; and
applying the cell-of-origin classification model to determine, based at least on the plurality of features extracted from the first PET scan, the first cell-of-origin associated with the first lesion.

15. (canceled)

16. (canceled)

17. (canceled)

18. The system of claim 1, further comprising:
receiving a computed tomography (CT) scan from a same timepoint as the first PET scan; identifying the first lesion depicted in the CT scan, wherein each pixel included in the CT scan is associated with an intensity value corresponding to a tissue density or an x-ray attenuation, and wherein each pixel in the first PET scan is associated with an intensity value corresponding to a level of metabolic activity;
determining, based on at least one of the CT scan and the first PET scan, a tumor mask corresponding to the first lesion;
determining, based on at least one of the CT scan and the first PET scan, an organ mask corresponding to one or more organs depicted in the CT scan and the first PET scan; and
applying the cell-of-origin classification model to determine, based at least on the tumor mask and the organ mask, the first cell-of-origin associated with the first lesion.

19. (canceled)

20. (canceled)

21. (canceled)

22. (canceled)

23. The system of claim 1, further comprising:
receiving a second positron emission tomography (PET) scan from a different timepoint as the first PET scan;
identifying the first lesion depicted in the second PET scan; and
applying the cell-of-origin classification model to determine, based on the first lesion depicted in the first PET scan and the second PET scan, the first cell-of-origin of the first lesion.

24. The system of claim 1, further comprising:
receiving a first computed tomography (CT) scan from a same timepoint as the first PET scan and a second CT scan from a same timepoint as the second PET scan;
identifying the first lesion depicted in the first CT scan and the first lesion depicted in the second CT scan; and
applying the cell-of-origin classification model to determine, based at least on the first lesion depicted in the first PET scan, the first CT scan, the second PET scan, and the second CT scan, the first cell-of-origin of the first lesion.

25. The system of claim 1, further comprising:
determining, based at least on the molecular subtype profile of the plurality of cancerous cells depicted in the first PET scan, a disease diagnosis, a disease prognosis, a disease progress, a treatment, and/or a treatment response for a patient associated with the first PET scan.

26. (canceled)

27. The system of claim 1, wherein the cell-of-origin classification model is trained to differentiate between germinal center B cell (GCB), non-germinal center B cell (non-GCB), and activated B cell (ABC).

28. (canceled)

29. (canceled)

30. The system of claim 1, wherein the cell-of-origin classification model includes at least one of an artificial neural network (ANN), a vision transformer, a vision transformer with shifted patch tokenization and locality self-attention, a tree-based classifier, or a ridge classifier.

31. (canceled)

32. (canceled)

33. (canceled)

34. (canceled)

35. The system of claim 1, further comprising:
training, based at least on a training set, the cell-of-origin classification model to determine, based on at least a portion of a positron emission tomography (PET) scan, a cell-of-origin of at least one lesion depicted in the PET scan.

36. The system of claim 35, further comprising:
extracting, from a second positron emission tomography (PET) scan, a first volume including a second lesion depicted in the second PET scan;
generating a first training sample to include the first volume and a first ground-truth annotation of a second cell-of-origin of the second lesion; and
generating the training set to include the first training sample.

37. The method of claim 36, further comprising:
generating, based at least on the first volume, a second volume by at least modifying the first volume; and
generating, for inclusion in the training set, a second training sample including the second
volume and the first ground-truth annotation of the second cell-of-origin of the second lesion.

38. (canceled)

39. The system of claim 37, wherein the modifying includes one or more of normalizing, rotating, flipping, and changing a zoom of the first volume

40. The system of claim 37, wherein the modifying of the first volume includes modifying one or more slices of the first volume that are within a threshold distance of a center of mass of the second lesion.

41. A computer-implemented method, comprising:
receiving a first positron emission tomography (PET) scan depicting a plurality of cancerous cells;
identifying a first lesion depicted in the first PET scan;
applying a cell-of-origin classification model to determine, based at least on the first lesion depicted in the first PET scan, a first cell-of-origin associated with the first lesion; and
determining, based at least on the first cell-of-origin of the first lesion, a molecular subtype profile for the plurality of cancerous cells depicted in the first PET scan.

42. A non-transitory computer readable medium storing instructions, which when executed by at least one data processor, result in operations comprising:
receiving a first positron emission tomography (PET) scan depicting a plurality of cancerous cells;
identifying a first lesion depicted in the first PET scan;
applying a cell-of-origin classification model to determine, based at least on the first lesion depicted in the first PET scan, a first cell-of-origin associated with the first lesion; and
determining, based at least on the first cell-of-origin of the first lesion, a molecular subtype profile for the plurality of cancerous cells depicted in the first PET scan.
